(19) United States
(12) Patent Application Publication
(10) Pub. No.: US 2024/0197498 A1
(43) Pub. Date: Jun. 20, 2024

(54) Devices for Preparation of a Bone Graft and Methods of Use Thereof

(71) Applicant: Stryker Corporation, Kalamazoo, MI (US)

(72) Inventors: Jeremy Ming Graul, Denver, CO (US); Kyle Craig Pilgeram, San Jose, CA (US)

(73) Assignee: Stryker Corporation, Kalamazoo, MI (US)

(21) Appl. No.: 18/543,480

(22) Filed: Dec. 18, 2023

Related U.S. Application Data
(60) Provisional application No. 63/433,633, filed on Dec. 19, 2022.

(51) Int. Cl. A61F 2/46; A61F 2/30; A61F 2/40

(52) U.S. Cl. CPC A61F 2/4644; A61F 2/30942; A61F 2/4081; A61F 2002/30955; A61F 2002/4648

(57) ABSTRACT

In an aspect, a clamp system is provided including a clamp and a base, the base including a graft-engaging block including a graft-engaging surface engaging a first surface of the graft, and the clamp contacts the graft while at least a portion of the graft opposite the first surface is removed. In another aspect, a method of preparing a distal radius bone graft is provided including positioning the distal radius bone graft into a clamp such that at least part of a proximal portion and at least another portion of the bone graft are accessible, performing a depth cut on the proximal portion of the graft to form a prepared proximal face, and performing a width cut on the another portion to form a prepared face.

### CROSS-REFERENCE TO RELATED APPLICATION

This application claims the benefit of the filing date of U.S. Provisional Patent Application No. 63/433,633, filed Dec. 19, 2022, the disclosure of which is hereby incorporated by reference herein in its entirety.

### BACKGROUND OF THE INVENTION

The glenohumeral joint of the shoulder is prone to issues causing discomfort and injury in patients. Issues include glenoid defects, critical bone loss, bony lesions, and sport collisions, which can cause anterior instability and even lead to chronic subluxation and dislocation in the shoulder. Critical bone loss, for instance, shortens the glenoid arc length, which reduces the size and concavity of the glenoid contact surface, decreasing stability. Patients suffering an initial glenoid dislocation, particularly at a young age, and patients having significant glenoid bone loss are likely to suffer recurring shoulder dislocations. Glenoid bone loss can be detected through physical evaluation with a physician and via radiographic evaluation, advanced imaging techniques, and arthroscopic measurements to precisely measure the defects.

Methods for repairing glenoid defects include soft-tissue stabilization and glenoid bone augmentation. Soft-tissue stabilization is suitable to repair minor defects and lesser amounts of bone loss. In patients with larger and more significant defects, or in patients for whom soft-tissue stabilization has not adequately restored the glenoid, bone augmentation may be required. The glenoid can be augmented with an autograft or allograft, such as in the Latarjet procedure using an autograft prepared from the coracoid bone block. After preparing the glenoid surface and the coracoid graft, the graft is affixed to the glenoid surface. The contacting surfaces of the glenoid and the coracoid graft are measured and prepared to match as closely as possible to ensure a stable fit. While the Latarjet procedure is successful for many patients, issues of stability often recur due at least in part to the imperfect fit of the coracoid graft against the glenoid as well as comorbidities associated with the use of the coracoid autograft such as axillary nerve injury. Recently, several bone sites have been considered for use in autografts and/or allografts, such as the distal tibia, medial tibia plateau, coracoid, iliac crest, distal clavicle, glenoid, radial head, and scapular spine. The distal tibia in particular offers high bone density and good articular conformity to the humeral head. Still, such allografts do not perfectly match the radius of curvature of the glenoid. Improved anatomic matching would provide improved stability, reducing glenohumeral osteoarthritis and injury recurrence.

Cutting jigs are frequently used in surgical environments to prepare bone for further surgical techniques and/or bone grafts to establish specific dimensions to fit a recipient site. A surgical technique guide frequently accompanies the cutting jig to instruct surgical operators how to properly use the jig in preparation of the graft. A common allograft in use today for this type of repair is a distal tibial allograft which offers articular cartilage and good radius of curvature matching in the superior/inferior direction, however this allograft has disadvantages. For instance, the distal tibia is relatively large compared to the graft needed for the glenoid repair, thereby requiring a large amount of cuts. Further, the distal tibia graft offers little radius of curvature in the medial/lateral direction and the distal tibia does not offer any accompanying soft tissue to reinforce the repair. A need exists, therefore, for (1) methods of glenoid augmentation with a graft closely tailored to the curvature of the glenoid to provide an improved contact area and increased stability and accompanying soft tissue for the repair and (2) a suitable cutting jig or clamp and surgical methods to prepare such a graft that can be simply used and to allow for a minimum number of cuts to be executed.

### BRIEF SUMMARY OF THE INVENTION

Generally, the present disclosure includes a cutting jig and a clamp, each for preparation of a graft, and methods of using the same.

In a first aspect, provided is a cutting jig for the preparation of a graft, comprising: a base component; a top component comprising an axial opening and a slot oriented radially about the top component, wherein the top component is rotatable about the base component; a base insert disposed within the axial opening and contacting at least one of the base component and the top component; and a cutting guide removably slidable within the slot.

The top component may further comprise a recessed surface having an adjustable height relative to the base component. Further, the slot may have an adjustable height relative to the base component. Furthermore, the adjustable height of the recessed surface and the adjustable height of the slot may be equal relative to the base component. Alternatively, the adjustable height of the recessed surface and the adjustable height of the slot may not be equal relative to the base component.

The top component may comprise a top set screw adjustable in a radial direction to contact the graft and prevent rotation of the top component about the base component. The top component may comprise a plurality of top set screws which may be positioned radially equidistant from one another. Each top set screw may be independently adjustable in a radial direction to contact the graft and prevent rotation of the top component about the base component.

The cutting guide may comprise a first set screw engageable with the graft and being adjustable to prevent movement of the cutting guide relative to the graft. The cutting guide may comprise a second set screw engageable with the top component and being adjustable to prevent movement of the cutting guide relative to the top component.

The base insert may have an upper face and a lower face and be disposed within the axial opening of the top component in a first orientation with the upper face facing toward the cutting guide or in a second orientation with the lower face facing toward the cutting guide. The base insert may include a graft-receiving area, the graft-receiving area including a graft-engaging surface configured to match a three-dimensional shape of a first surface of the graft. In an aspect, the first surface is a dorsal surface and the graft-engaging surface is a dorsal-engaging surface. In another aspect, the first surface is a palmar surface and the graft-engaging surface is a palmar-engaging surface. Furthermore, the base insert may be formed using a 3D printing process such that the graft-engaging surface matches the three-dimensional shape of the first surface of the graft. Or the graft-engaging surface may match a common surface of a plurality of grafts. Or the graft-engaging surface may have mating features based on average curvatures and dimensions of one or more graft surfaces determined from a database. Further, the graft-engaging surface may comprise a plurality of modular inserts configured to match the average curvatures and dimensions of the one or more graft surfaces. Or the base insert may comprise a graft-receiving area including a graft-engaging surface having a V-block shape. Or the base insert may comprise a graft-receiving area including a graft-engaging surface having a U-block shape. The graft-receiving area of the base insert may be shaped to contact a distal radius bone graft at a scaphoid facet and at a lunate facet to increase stability and alignment of the distal radius bone graft relative to the cutting jig. The base insert may comprise a flippable graft-receiving area. The base insert may comprise a flat proximally-engaging surface. Or the base insert may comprise a proximally-engaging surface shaped to match a proximal portion of the distal radius bone graft.

The cutting jig may have a cylindrical shape. The cutting jig may be configured to manipulate and secure a distal radius bone graft.

In a second aspect, provided is a method of preparing a distal radius bone graft comprising: (S1) obtaining the distal radius bone graft; (S2) positioning the distal radius bone graft into a cutting jig or a clamp such that a first surface of the distal radius bone graft is removably coupled to a graft-engaging surface of the cutting jig or the clamp and at least part of a proximal portion and at least part of a portion opposite the first surface of the distal radius bone graft are accessible; (S3) performing a depth cut on and approximately parallel to the proximal portion of the graft to form a prepared proximal face; and (S4) performing a width cut on and approximately parallel to the portion opposite the first surface of the graft to form a prepared face. The steps (S3) and (S4) may be performed in either order as desired.

In an aspect, the first surface is a dorsal surface, the portion opposite the first surface is a palmar portion, the prepared face is a prepared palmar face, and the graft-engaging surface is a dorsal-engaging surface. In another aspect, the first surface is a palmar surface, the portion opposite the first surface is a dorsal portion, the prepared face is a prepared dorsal face, and the graft-engaging surface is a palmar-engaging surface.

In an aspect, the method further comprises, prior to step S3, performing a rough depth cut on at least part of and approximately parallel to the proximal portion of the graft to separate the graft from a remainder of a distal radius and to form a rough proximal face of the graft. In a further aspect, the rough depth cut is performed prior to step S1. In an aspect, the rough depth cut is performed while the graft is positioned in the cutting jig or the clamp.

In an aspect, the method further comprises, prior to step S4, performing a rough width cut on at least part of and approximately parallel to the portion opposite the first surface of the graft to remove a cornice of bone on the portion opposite the first surface and to form a rough face of the graft. In an aspect, the rough face of the graft is a rough palmar face. In another aspect, the rough face of the graft is a rough dorsal face. In a further aspect, the rough width cut is performed prior to step S1. In an aspect, the rough width cut is performed while the graft is positioned in the cutting jig or the clamp.

In an aspect, the cutting jig comprises a base component, a top component comprising an axial opening, and a base insert, wherein the base insert is disposed within the axial opening and contacts at least one of the base component and the top component, wherein the base insert comprises a flippable graft-receiving area, and wherein the method further comprises, prior to step S4, flipping the graft-receiving area to expose a distal face of the graft to a surgical operator.

In an aspect, the method further comprises, after step S2 and prior to step S4: removing the graft from the clamp; and repositioning the graft into the clamp such that the first surface of the graft is removably coupled to the graft-engaging surface of the clamp and such that the graft is flipped to expose a distal face of the graft to a surgical operator. In a further aspect, the clamp comprises a first securement feature and a second securement feature, the removing comprises disengaging each of the first securement feature and the second securement feature from the graft, and the repositioning comprises engaging each of the first securement feature and the second securement feature with the graft to secure the graft within the clamp. In still a further aspect, prior to removing the graft from the clamp, the first securement feature contacts the graft at a scaphoid facet and the second securement feature contacts the graft at a lunate facet, and after repositioning the graft into the clamp, the first securement feature contacts the graft at the lunate facet and the second securement feature contacts the graft at the scaphoid facet. In an aspect, the first securement feature comprises a first prong and the second securement feature comprises a second prong.

In an aspect, the method further comprises, prior to steps S3 and S4, determining average curvatures and dimensions of one or more glenoid surfaces for each of a plurality of glenoids from a database, wherein steps S3 and S4 are performed such that curvatures and dimensions of the prepared proximal face and the prepared face match the average curvatures and dimensions of the one or more glenoid surfaces. In an aspect, the method further comprises, prior to steps S2, S3, and S4, determining average curvatures and dimensions of one or more graft surfaces from a database, wherein steps S2, S3, and S4 are performed with the cutting jig or the clamp comprising a graft-engaging surface with mating features based on the average curvatures and dimensions of the one or more graft surfaces. In a further aspect, the graft-engaging surface comprises a plurality of modular inserts configured to match the average curvatures and dimensions of the one or more graft surfaces.

In an aspect, the cutting jig described herein with regard to the method is the cutting jig described above. In an aspect, the clamp described herein with regard to the method is the clamp described in other aspects herein.

In an aspect, the cutting jig comprises a top component and a base component, the top component including a top set screw adjustable in a radial direction to contact the distal radius bone graft and prevent rotation of the top component about the base component and step S2 further comprises adjusting the top set screw to prevent rotation of the top component about the base component.

In another aspect, the clamp comprises a first securement feature and a second securement feature, and wherein step S2 further comprises engaging each of the first securement feature and the second securement feature with the graft to secure the graft within the clamp. In a further aspect, the portion opposite the first surface does not contact any of the clamp, the first securement feature, and the second securement feature. In still a further aspect, step S4 further comprises continuously securing the graft in the clamp while performing the width cut.

In another aspect, step S2 further comprises aligning the proximal portion relative to a slot oriented radially about a top component of the cutting jig. In a further aspect, step S2 further comprises removing a cutting guide via the slot. In still a further aspect, step S3 further comprises passing a resection tool through the slot. In still a further aspect, step S3 further comprises moving a resection tool approximately along a recessed surface of the top component.

In an aspect, step S2 further comprises aligning the portion opposite the first surface relative to at least one of a slot oriented radially about a top component of the cutting jig, a cutting guide removably slidable within the slot, a base insert disposed within an axial opening and contacting at least one of the base component and the top component, and the axial opening of the top component. In a further aspect, step S4 further comprises aligning the cutting guide relative to the prepared face. In still a further aspect, step S4 further comprises passing a resection tool through the axial opening relative to the cutting guide.

In an aspect, the method comprises, subsequent to step S3, positioning the portion opposite the first surface relative to at least one of a slot oriented radially about a top component of the cutting jig, a cutting guide removably slidable within the slot, and an axial opening of the top component. In a further aspect, step S4 further comprises aligning the cutting guide relative to the prepared face. In still a further aspect, step S4 further comprises passing a resection tool through the axial opening relative to the cutting guide. In an aspect, the cutting guide comprises a first set screw engageable with the distal radius bone graft and being adjustable to prevent movement of the cutting guide relative to the distal radius bone graft and step S2 further comprises engaging the first set screw to prevent movement of the cutting guide relative to the distal radius bone graft. In a further aspect, the cutting guide comprises a second set screw engageable with the top component and being adjustable to prevent movement of the cutting guide relative to the top component and step S2 further comprises engaging the second set screw to prevent movement of the cutting guide relative to the top component.

In an aspect, the cutting jig comprises a base component, a top component comprising an axial opening and a slot oriented radially about the top component, wherein the top component is rotatable about the base component, and a cutting guide removably slidable within the slot, and step S2 further comprises rotating the top component relative to the base component and at least partially retracting or inserting the cutting guide relative to the slot.

In an aspect, the method further comprises: assessing a fit of the distal radius bone graft relative to a glenoid; and repeating steps S2, S3, and S4 as necessary to revise the fit.

In an aspect, the method further comprises drilling a plurality of channels into the graft. In a further aspect, the clamp comprises a drilling guide comprising a plurality of channels, and the drilling comprises forcibly pressing a drill bit through the plurality of channels of the drilling guide to contact the graft and form a plurality of channels within the graft, each channel within the graft being aligned to a channel of the drilling guide. In still a further aspect, the clamp further comprises a graft-engaging block comprising: a graft-engaging surface configured to engage the first surface of the graft and secure the graft within the clamp, and a plurality of channels, each channel of the graft-engaging block being aligned to a channel of the drilling guide, wherein step S2 further comprises engaging the first surface of the graft with the graft-engaging surface to secure the graft within the clamp. In an aspect, the method further comprises, prior to the drilling, positioning the drilling guide against the portion opposite the first surface of the graft.

In an aspect, each of steps S3 and S4 is performed while the graft is positioned in the jig or the clamp.

Provided in a third aspect is a clamp for the preparation of a distal radius bone graft, comprising: a securing tool configured to contact the graft and secure the graft within the clamp; and a base connected to the securing tool, the base comprising a graft-engaging block; wherein the graft-engaging block comprises a graft-engaging surface configured to engage a first surface of the graft and secure the graft within the clamp, wherein the securing tool is configured to continuously contact and secure the graft while at least part of a portion opposite the first surface of the graft is removed from the graft.

In an aspect, the securing tool comprises a first securement feature and a second securement feature configured to contact and secure the graft, wherein in a first configuration, the first securement feature is configured to contact a scaphoid facet of the graft and the second securement feature is configured to contact a lunate facet of the graft, and wherein in a second configuration, the first securement feature is configured to contact the lunate facet of the graft and the second securement feature is configured to contact the scaphoid facet of the graft. In an aspect, the first securement feature comprises a first prong and the second securement feature comprises a second prong. In a further aspect, the first securement feature comprises a plurality of first securement features and the second securement feature comprises a plurality of second securement features. In an aspect, the securing tool further comprises: a first end connected to the first securement feature; a second end connected to the second securement feature; and a fulcrum housing connecting the first end and first securement feature to the second end and second securement feature, wherein the first end and the second end are configured such that when the first end and the second end are moved toward one another, the first securement feature and the second securement feature are moved toward one another, and vice versa. In a further aspect, the securing tool further comprises a ratchet configured to maintain a set distance between the first securement feature and the second securement feature. In still a further aspect, the first securement feature, the second securement feature, the first end, the second, and the ratchet are together configured to continuously secure the graft within the clamp without continuous or repeated physical input from a surgical operator. In still a further aspect, the first securement feature, the second securement feature, the first end, the second end, and the ratchet are together configured to continuously secure the graft within the clamp without contacting a portion opposite the first surface of the graft. In an aspect, the base further comprises a fulcrum channel configured to connect the base to the securing tool and oriented such that the securing tool can be moved toward or away from the graft by moving along the fulcrum channel.

In an aspect, the graft-engaging surface comprises a V-block shape. In an aspect, the graft-engaging surface is configured to match a three-dimensional shape of a first surface of the graft. In an aspect, the first surface is a dorsal surface, the portion opposite the first surface is a palmar portion, and the graft-engaging surface is a dorsal-engaging surface. In another aspect, the first surface is a palmar surface, the portion opposite the first surface is a dorsal portion, and the graft-engaging surface is a palmar-engaging surface. In an aspect, the graft-engaging surface is formed using a 3D printing process such that the graft-engaging surface matches a three-dimensional shape of a first surface of the graft. Or, the graft-engaging surface may comprise mating features based on average curvatures and dimensions of one or more graft surfaces determined from a database. Further, the graft-engaging surface may comprise a plurality of modular inserts configured to match the average curvatures and dimensions of the one or more graft surfaces.

In an aspect, the base comprises a drilling guide comprising a plurality of guide channels, the drilling guide being configured to drill a plurality of channels through the graft when a drill bit is passed through the plurality of guide channels and forcibly pressed against the graft. In a further aspect, the base further comprises a guide-engaging channel configured to connect the drilling guide to the base and oriented such that the drilling guide can be moved toward or away from the graft by moving along the guide-engaging channel.

In an aspect, the securing tool further comprises a first securement feature and a second securement feature, and wherein the first securement feature, the second securement feature, the base, and the graft-engaging block are together configured to contact and secure the graft within the clamp.

In an aspect, the securing tool comprises a first securement feature and a second securement feature configured to continuously contact and secure the graft, without contacting a portion opposite the first surface of the graft such that at least part of the portion opposite the first surface of the graft may be removed without physical interference from the first securement feature and the second securement feature.

In another aspect, provided is a method of preparing a distal radius bone graft consisting of: obtaining the distal radius bone graft; positioning the distal radius bone graft into a cutting jig or a clamp such that a first surface of the distal radius bone graft is removably coupled to a graft-engaging surface of the cutting jig or the clamp and at least part of a proximal portion and at least part of a portion opposite the first surface of the distal radius bone graft are accessible; performing a depth cut on and approximately parallel to the proximal portion of the graft to form a prepared proximal face of the graft; and performing a width cut on and approximately parallel to the portion opposite the first surface of the graft to form a prepared face of the graft.

In another aspect, provided is a method of preparing a distal radius bone graft consisting of: obtaining the distal radius bone graft; positioning the distal radius bone graft into a cutting jig or a clamp such that a first surface of the distal radius bone graft is removably coupled to a graft-engaging surface of the cutting jig or the clamp and at least part of a proximal portion and at least part of a portion opposite the first surface of the distal radius bone graft are accessible; performing a rough depth cut on at least part of and approximately parallel to the proximal portion of the graft to separate the graft from a remainder of a distal radius and to form a rough proximal face of the graft; performing a depth cut on and approximately parallel to the proximal portion of the graft to form a prepared proximal face of the graft; performing a rough width cut on at least part of and approximately parallel to the portion opposite the first surface of the graft to remove a cornice of bone on the portion opposite the first surface and to form a rough face of the graft; and performing a width cut on and approximately parallel to the portion opposite the first surface of the graft to form a prepared face of the graft.

In another aspect, provided is a clamp system for the preparation of a graft including a clamp including a securement device configured for securing the graft, and a base configured for including a graft-engaging surface, and a plurality of modular inserts configured to match the average curvatures and dimensions of one or more surfaces of the graft.

### BRIEF DESCRIPTION OF THE DRAWINGS

FIGS. 1-8 and 10 show various elements of the cutting jig for preparation of a graft.

FIG. 9 shows the graft.

FIGS. 10-14 show various stages of a method for preparing a graft.

FIGS. 15 and 16 show a prepared graft aligned to a patient glenoid.

FIGS. 17-22 show various elements of the clamp for preparation of a graft and show various stages of a method for preparing a graft.

### DETAILED DESCRIPTION

As used herein, the terms “about,” “generally,” and “substantially” are intended to mean that slight deviations from absolute are included within the scope of the term so modified. The term “match” means to approximately match to an extent; for instance, when referring to matching of the graft to one or more surfaces or portions of a glenoid, the term means that the graft is suitable to replace a portion of the glenoid, within acceptable design and cutting tolerances as determined by a surgical operator.

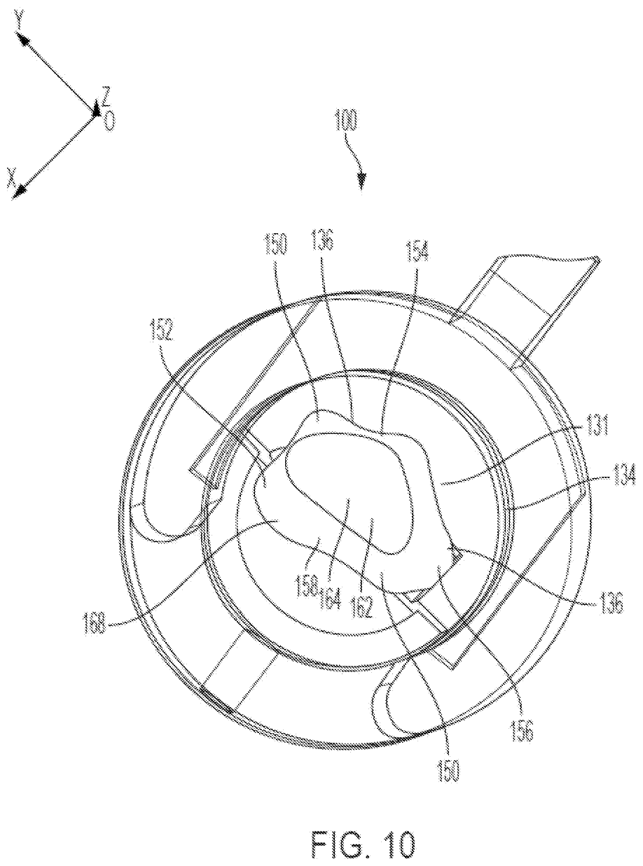

Many of the aspects and examples discussed herein refer to cuts performed along a certain axis, plane, or face, or cuts performed in a certain direction. For example, as discussed below with reference to FIG. 10, the rough depth cut is performed in a direction along the z-axis of the graft.

It is to be understood herein, and one of ordinary skill in the art would readily appreciate, that when referring to one or more cuts along a certain axis, plane, or face, or in a certain direction, that such cut or cuts are not limited to a precise angle or direction, but rather the axis, plane, face, or direction indicates a general direction of the cut. Accordingly, cuts “along” an axis, place, or face, or “in” a certain direction do not necessarily form an angle of 0° with the axis, plane, face, or direction. Furthermore, cuts described as “perpendicular,” “orthogonal,” or “normal” to an axis, plane, face, or direction do not necessarily form an angle of 90° with the axis, plane, face, or direction. Similarly, objects and/or features described as “aligned,” “parallel,” or “perpendicular” to one another are to be understood as being generally aligned, parallel, or perpendicular to one another and are not limited to a precise angle or an exact alignment. As used herein, such directional language may be modified by terms such as “approximately” (e.g., approximately parallel) to further illustrate such allowances beyond the strict direction—e.g., a cut being approximately or generally parallel rather than strictly parallel to a relative axis, surface, face, or the like. For example, a cut or member with orientation described as “along,” “in,” “perpendicular,” “orthogonal,” or “normal” to an axis, plane, face, or direction, or “approximately” thereto, may indicate a deviation within 5° of the precise orientation, or within 10°, or within 15°, or within 20°, or within 25°, or within 30°, or may indicate any suitable deviation from the precise orientation as understood by those of ordinary skill in the art.

The inventors believe the distal radius to be a beneficial allograft option to repair the glenoid for four main reasons. First, the distal radius includes articular cartilage. Second, the curvature of the distal radius closely matches that of the glenoid in both the superior/inferior direction as well as the anterior/posterior direction. Third, the overall size of the distal radius is similar to that of the glenoid, so the preparation and number of resections to prepare the allograft are reduced. Lastly, the distal radius allograft includes integrated soft tissue (carpal ligaments) that can be incorporated into the repair.

Many of the aspects and examples discussed herein refer specifically to a distal radius bone graft, which is the preferred graft source material, but any graft made of a bone block, bone plug, artificial material, or any other suitable material or combination thereof may be used in place of the distal radius graft, and the cutting jig may be used so long as it is appropriately configured for accommodating such an alternatively sourced graft material. Alternative anatomical donor sites may include the distal tibia, medial tibia plateau, coracoid, iliac crest, distal clavicle, glenoid, radial head, and scapular spine, for example, or can be from another anatomical location. Such a graft may be autologous tissue (autograft) taken from the patient, though more likely the graft may be from a donor (allograft) or may be from a donor of another species (xenograft). Further, as to the use of a distal radius bone graft, many of the embodiments illustrated and discussed herein illustrate preparation (e.g., cuts) being performed on the palmar and proximal surfaces of the graft. However, additional or alternative surfaces can be prepared. For instance, the dorsal surface of the graft may be prepared (e.g., cut) in lieu of the palmar surface to shape the graft for use. Indeed, while the preparations illustrated herein are preferred, the inventors recognize that there may be benefits to instead prepare the dorsal surface of the distal radius graft as such preparations may serve to remove portions of the bone graft considered to be weaker than other bony portions of the graft.

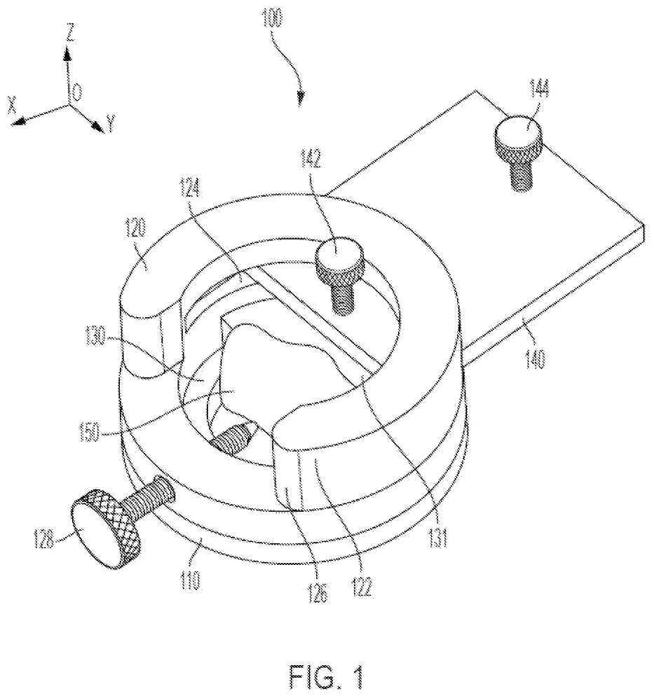

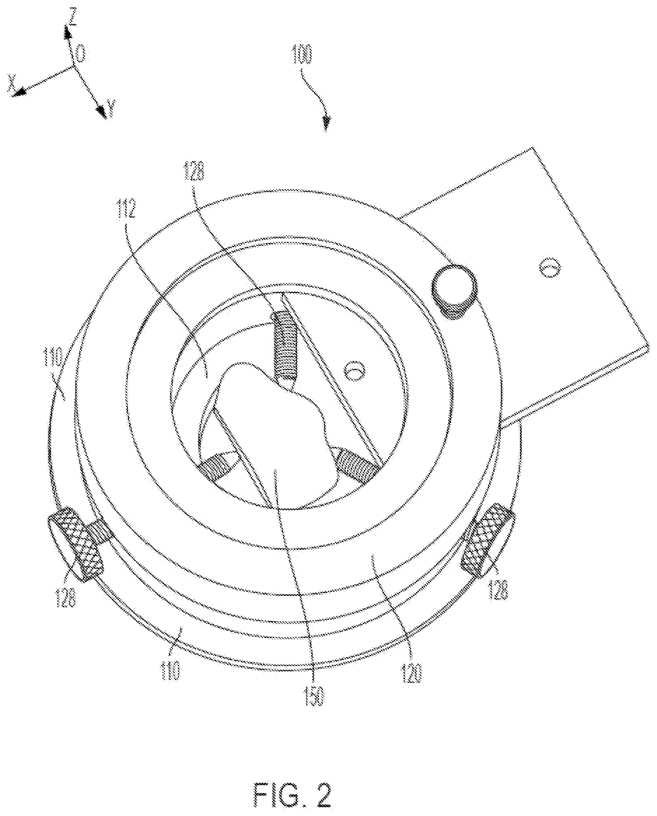

FIGS. 1-8 and 10 show a cutting jig 100 according to an exemplary aspect. As shown in FIG. 1 in particular, the cutting jig 100 may have a cylindrical shape. The cutting jig 100 includes a base component 110 and a top component 120 and is configured to manipulate and secure a distal radius bone graft 150, which can be inserted into an axial opening 122 and secured against a base insert 130 with a top set screw 128. The top component 120 is disposed on and is rotatable about the base component 110. The top component 120 includes the axial opening 122. Part or all of the axial opening 122 may persist through the entire cutting jig 100 as shown in FIG. 1. Alternatively, as shown in FIG. 2, the base component 110 may be closed at the bottom such that the axial opening 122 only persists through the top component 120.

The top component 120 further includes a slot 124 oriented radially about the top component 120. The slot 124 partially circumnavigates the top component 120. The slot 124 is configured to accept a cutting guide 140, which is removably slidable in and out of the slot 124. The slot 124 is positioned within top component 120 at a height relative to the base component 110, though this height may be adjustable as desired, as discussed further below. The top component 120 further includes a recessed portion 126 which, like the slot 124, is part of top component 120 and is at a height relative to the base component 110, though this height may be adjustable as desired, as also discussed further below. In an aspect, the adjustable height of the slot 124 and the adjustable height of the recessed portion 126 are equal to one another, relative to the base component, and may be adjusted simultaneously or individually. In another aspect, the adjustable height of the slot 124 and the adjustable height of the recessed portion 126 are not equal to one another, relative to the base component, and may be adjusted simultaneously or individually. The adjustable height of the slot 124 may be achieved by including multiple slots 124 each having different heights relative to the base component 110. Or, the adjustable heights of the slot 124 and the recessed portion 126 may be adjusted by inserting opposing plates into each of the slot 124 and the recessed portion 126 to create a sloped plane between the slot 124 and the recessed portion 126. Or, one or more adjustable set screws (not shown) may be included in the cutting jig 100 in order to adjust the height of the slot 124, the recessed portion 126, or both. Or, the height adjustments to the slot 124 and the recessed portion 126 may be performed in any suitable manner known to those of ordinary skill in the art. Such adjustments may be performed on the slot 124 and the recessed portion 126 simultaneously or individually. Such adjustments may be performed on a single slot 124 or multiple slots 124, each having different heights relative to the base component 110.

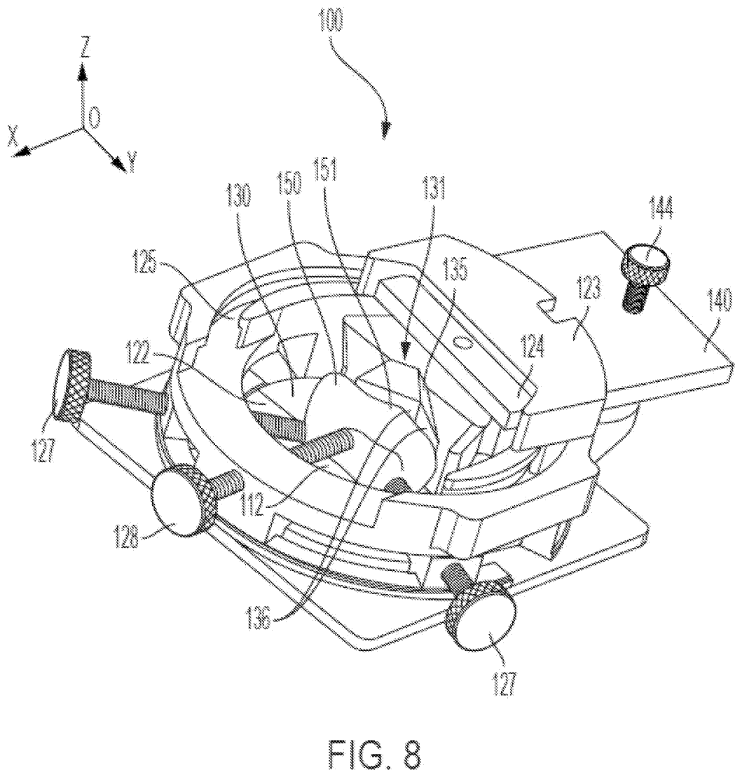

The top component 120 further includes the top set screw 128 which is adjustable in a radial direction to contact other elements relative to cutting jig 100, such as the graft 150, which may limit or prevent rotation of the top component 120 relative to the base component 110. As illustrated in FIG. 1, tightening the top set screw 128 secures both the graft 150 and the top component 120 against the base insert 130, which thus limits or prevents relative rotation with base component 110. In the exemplary aspect shown in FIG. 1, the top set screw 128 is a single screw. Alternatively, as shown in FIG. 2, the top component 120 may include a plurality of top set screws 128 positioned radially (as illustrated for example, equidistant) from one another and each being adjustable in a radial direction to contact the graft 150 and prevent rotation of the top component 120 about the base component 110. Further alternatively, as shown in FIG. 8 and discussed below, the top component 120 may include a center top set screw 128 and two outer top set screws 127 positioned radially about the top component 120. A plurality of top set screws 128 allows for a uniform distribution of pressure on the graft 150 which may further ensure stability and may prevent potential erosion of the graft 150 from the applied force of a single top set screw 128.

A base insert 130 is disposed within the axial opening 122 and contacts at least one of the base component 110 and the top component 120. The base insert 130 includes a graft-receiving area 131. In an aspect, the graft-receiving area 131 includes a removable surface 112 having a half circle or a full circle shape (full circle shown intact in FIG. 2, not shown in FIG. 1), which is discussed further below with regard to FIG. 8.

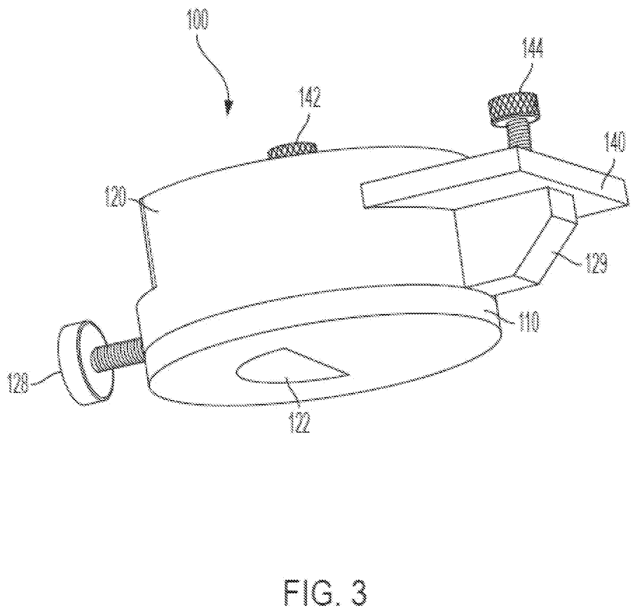

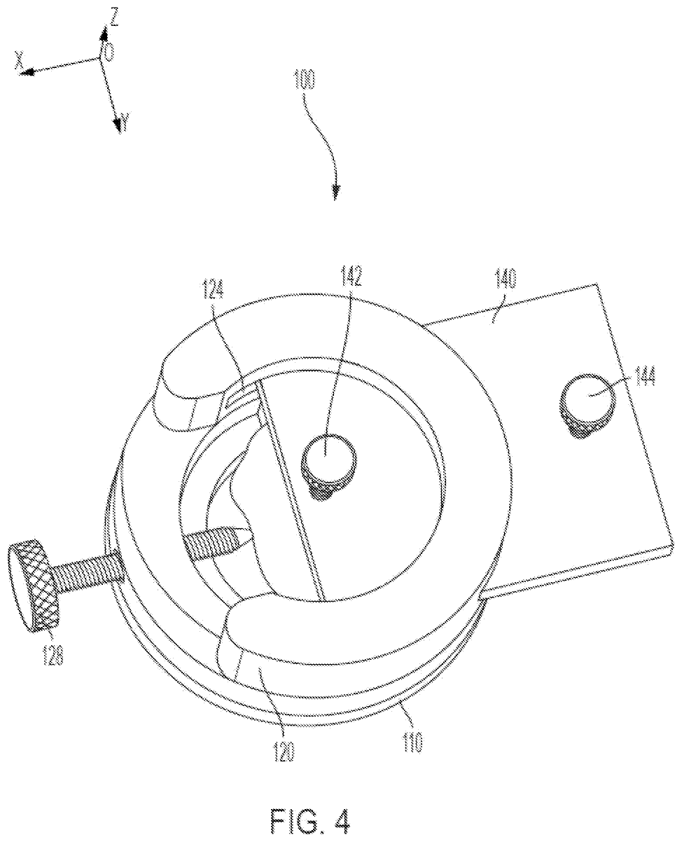

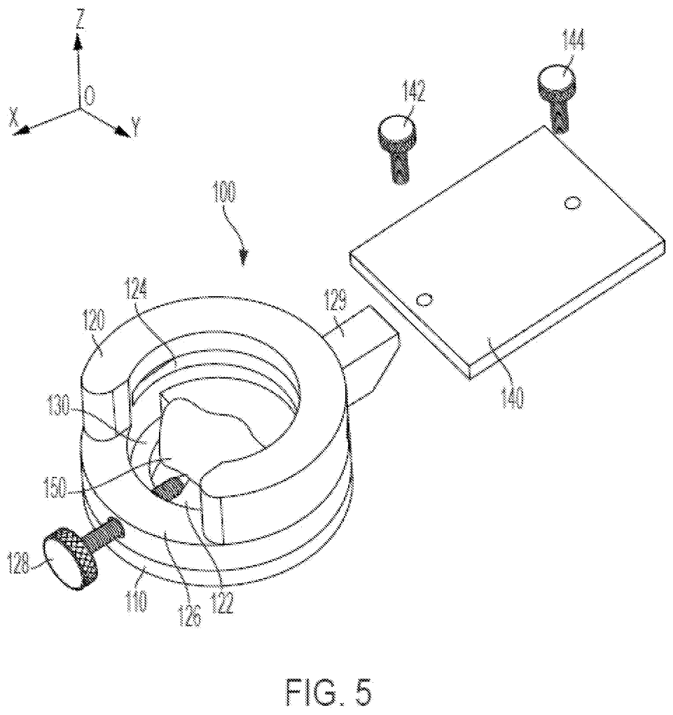

The cutting guide 140 includes a first set screw 142 engageable with another element, such as for example the graft 150, and adjustable to limit or prevent movement of the cutting guide 140 relative to the graft 150. The cutting guide 140 may further include a second set screw 144 engageable with another element, such as for example the top component 120, and adjustable to limit or prevent movement of the cutting guide 140 relative to the top component 120. More specifically and as shown in FIG. 3, the second set screw 144 may be engageable with a platform 129 of the top component 120 and is adjustable to, once tensioned, limit or prevent movement of the cutting guide 140 relative to platform 129 of the top component 120. As shown in FIG. 4, a loosening adjustment of the top set screw 128 would allow for rotation of the top component 120 about the base component 110 since the top set screw would release pressure on graft 150, thereby allowing top component 120 to move relative to the graft and base component 110. As further shown in FIG. 4, a loosening adjustment of the first set screw 142 and, if present, the second set screw 144 would allow for slidable retraction of the cutting guide 140 with respect to the slot 124. FIG. 5 shows a complete loosening of the first set screw 142 and the second set screw 144 and complete removal of the cutting guide 140 from the slot 124.

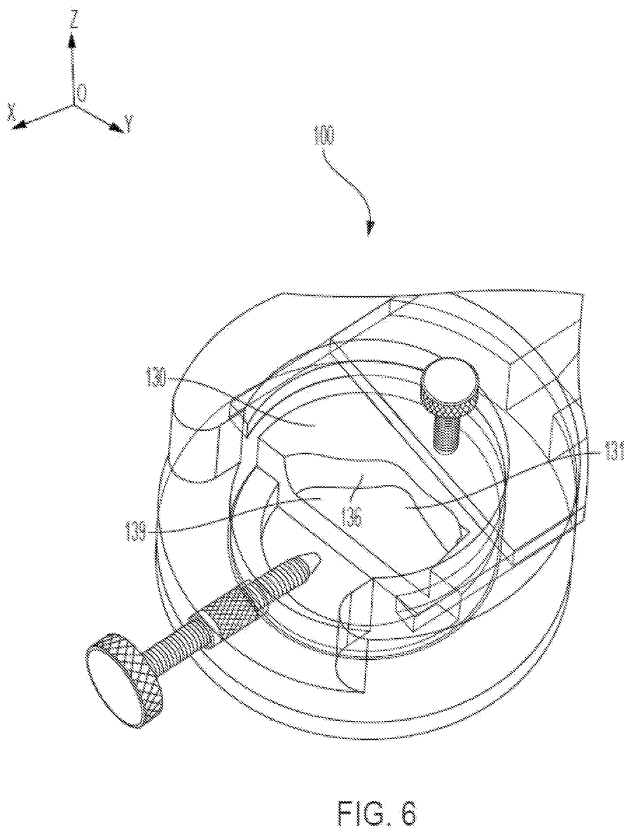

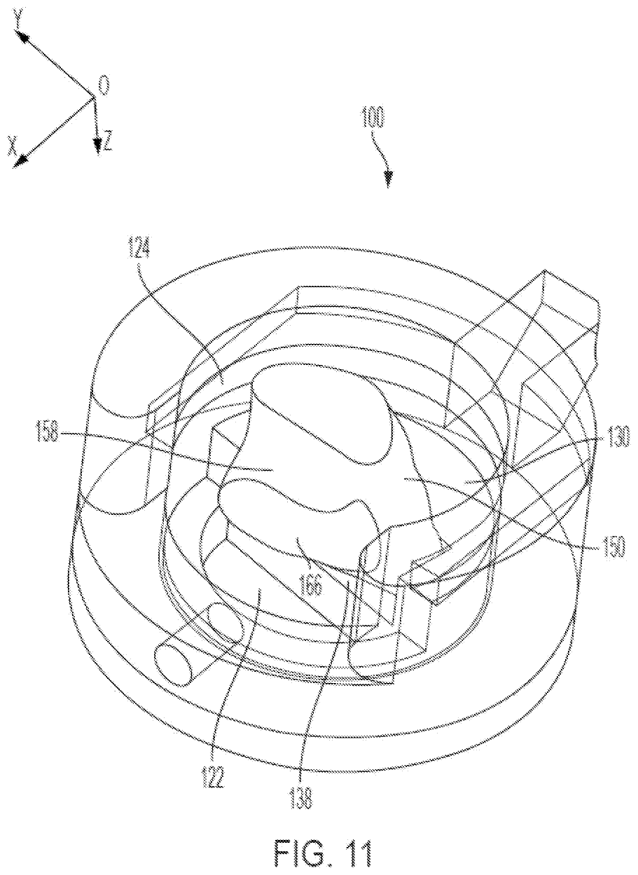

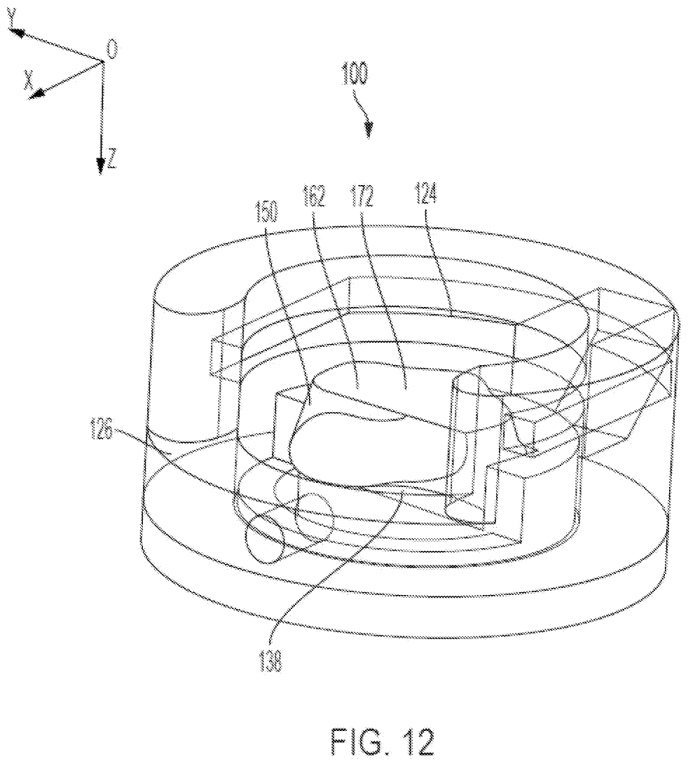

FIG. 6 shows one aspect of the base insert 130 further including a graft-receiving area 131. The graft-receiving area 131 includes a graft-engaging surface 136, a proximally-engaging surface 139, and a distally-engaging surface 138 (shown in FIGS. 11 and 12). In an aspect, as shown in FIG. 6, the proximally-engaging surface 139 is a flat bottom surface 139. In an aspect, as shown in FIG. 6, the graft-engaging surface 136 is configured to match a three-dimensional shape of the graft 150. A graft-engaging surface 136 according to this aspect may be patient-specific. Thus, the graft-engaging surface 136 according to this aspect is not necessarily symmetrical and is specific to the complex geometry of the graft 150. The graft-engaging surface 136 according to this aspect may be prepared by conforming the surface 136 to the three-dimensional shape of the graft 150 by any suitable method. For instance, such preparation may involve determination of the graft-engaging surface 136 by measuring dimensions and curvatures of one or more surfaces of the donor's distal radius prior to acquisition of the distal radius graft 150.

Determination of the graft-engaging surface 136 may further or alternatively include measuring dimensions and curvatures of one or more surfaces of the recipient patient's glenoid prior to or after acquisition of the distal radius graft 150. Such measurements may be made by any suitable methods known in the art, such as with a computed tomography (CT) scan. Thickness of cartilage may be accounted for in order to prepare the properly-sized graft-engaging surface 136.

Determination of the graft-engaging surface 136 may further or alternatively include preparing an average surface of the donor's distal radius by analyzing multiple distal radii in a lookup table or a database, such as the Stryker Orthopedic Modeling and Analytics Anatomic Database and Tools (“SOMA”) (Stryker Corporation, Mahwah, NJ). Particularly, the graft-engaging surface 136 may include mating features based on average curvatures and dimensions of one or more graft surfaces determined from a database such as SOMA. Such databases include scans and dimensions of anatomy for numerous patients and allow for filtering by ethnicity, sex, age, height, and other factors. Average surface dimensions and curvatures of the distal radius can thus be prepared by averaging measurements and curvatures of a filtered selection of patients in the database whose physical features closely match those of the donor. Similarly, determination of the graft-engaging surface 136 may include preparing an average surface of the recipient's glenoid using a database as just described. In an aspect, the graft-engaging surface 136 matches a common surface of a plurality of grafts. Though not shown, the graft-engaging surface 136 may include a plurality of modular inserts configured to match the average curvatures and dimensions of one or more graft surfaces. Specifically, the plurality of modular inserts may be configured to match the average curvatures and dimensions of any one or more of the first surface 154, the surface 158 which is opposite the first surface 154, the lunate facet 152, the scaphoid facet 156, the proximal portion 162, or the distal face 176. Once the graft-engaging surface 136 has been determined, the surface 136 can be manufactured according to any suitable method known in the art, for example additive manufacturing processes such as 3D printing. In an aspect, the base insert 130 is formed using a 3D printing process such that the graft-engaging surface 136 matches the three-dimensional shape of the first surface 154 of the graft 150 (first surface 154 being shown in FIG. 10).

The above discussion regarding the graft-engaging surface 136 is equally applicable to the distally-engaging surface 138 and to the proximally-engaging surface 139. That is, surfaces 138 and 139 may each be shaped to match one or more surfaces of the graft 150. The surfaces 138 and 139 may each independently be patient-specific or have a generic shape. The surfaces 138 and 139 may be determined by measuring dimensions and curvatures of one or more surfaces of the donor's distal radius, measuring dimensions and curvatures of one or more surfaces of the recipient patient's glenoid, preparing an average surface of the donor's distal radius by analyzing multiple distal radii in a lookup table or a database, or other suitable methods known in the art, and the patient-specific surfaces 138 and 139 may be prepared by 3D printing or other suitable methods known in the art. Further, surfaces 138 and 139 may each independently include a plurality of modular inserts configured to match the average curvatures and dimensions of one or more graft surfaces.

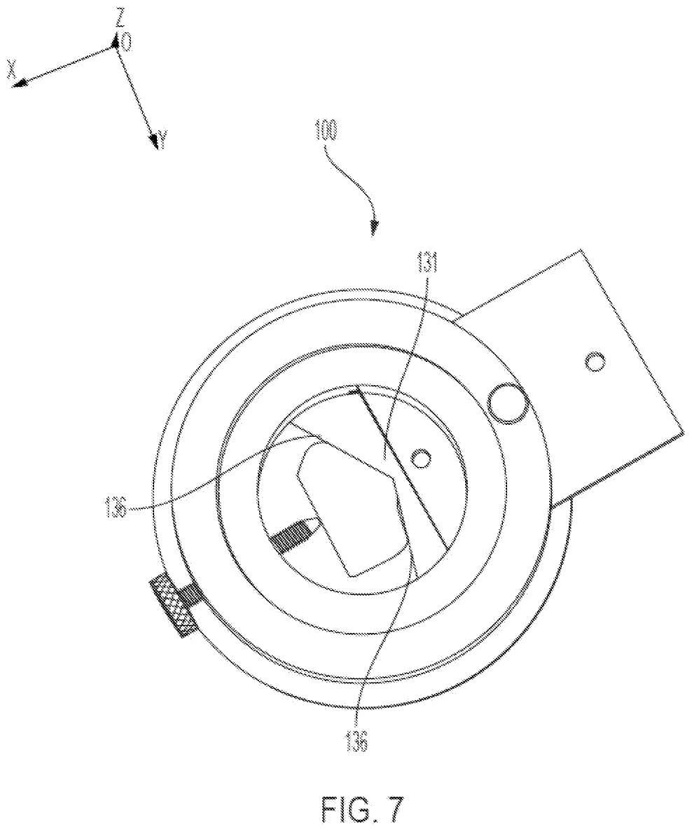

A precise match between the three-dimensional shape of the graft 150 and surface 136 is not required, and thus surface 136 may have other shapes suitable to stabilize the graft 150 within the cutting jig 100. In an aspect, as shown in FIG. 7, the graft-engaging surface 136 has a generic V-block shape including two surfaces 136 forming the V-block shape. In another aspect (not illustrated), the graft-engaging surface 136 of the graft-receiving area 131 has a generic U-block shape, or may have another generic geometric shape. The graft-engaging surface 136 according to such aspects is a concave surface having two portions that may or may not form a symmetrical surface. Still further, surface 136, as well as or alternatively proximally engaging-surface 139 and distally-engaging surface 138 (shown in FIGS. 6 and 12, respectively), may include surface textures that assist in stabilizing the graft 150, such as serrations, roughened textures, tacky or sticky coatings, or the like.

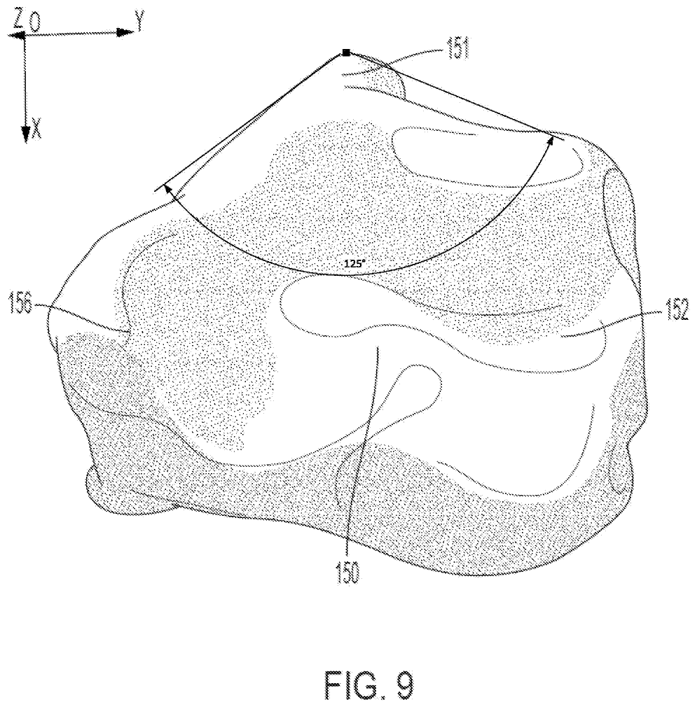

FIG. 8 shows the cutting jig 100 according to one or more aspects. In an aspect and as shown, the graft-receiving area 131 may have a V-block shape including two graft-engaging surfaces 136 at an angle of approximately 120°, and the graft-receiving area 131 may further include an overhanging surface 135. Such a graft-receiving area 131 is configured to accept the graft 150. Particularly, the one or more graft-engaging surfaces 136 are configured to engage the first surface 154 of the graft 150. In particular to this illustrated embodiment, the dorsal-engaging surfaces 136 are configured to accept the Lister's tubercle 151 when the dorsal surface 154 (not visible in FIG. 8) of graft 150 is pressed into the graft-receiving area 131 against the dorsal-engaging surfaces 136. As shown in FIG. 9, when the scaphoid facet 156 is on the left and the lunate facet 152 is on the right, the Lister's tubercle 151 forms a V-shape with, as illustrated in this example, an angle of approximately 125° which fits into the graft-receiving area 131. Though not explicitly shown in FIG. 9, the Lister's tubercle 151 forms a similar V-shape that fits into the graft-receiving area 131 when the graft 150 is flipped relative to the orientation shown in FIG. 9 such that the distal face 176 is facing down while the lunate facet 152 is on the left and the scaphoid facet 156 is on the right. Further, the graft-receiving area 131 includes an overhanging surface 135 to secure the graft 150. The overhanging surface 135 prevents the graft 150 from moving upward due to upward compression from the top set screw 128 and/or the two outer top set screws 127. The overhanging surface 135 is particularly useful to prevent upward movement of the graft 150 when the graft 150 is facing downward.

In an aspect and as shown in FIG. 8, the top component 120 may include a center top set screw 128 and two outer top set screws 127. According to this aspect, the two outer top set screws 127 are slidable circumferentially about the top component 120 such that each outer top set screw 127 forms an angle with the center top set screw 128 between about 10° and about 90°. The center top set screw 128 and outer top set screws 127 are each adjustable in a radial direction to contact the graft 150 and prevent rotation of the top component 120 about the base component 110. The center top set screw 128 and outer top set screws 127 may be adjusted in various configurations in order to stabilize the graft 150 and allow space for cutting the graft 150. For example, the outer top set screws 127 may be adjusted to engage and secure the graft 150 while the center top set screw 128 is adjusted so as not to contact the graft and to be partially removed from the top component 120 in order to increase the open space of the axial opening 122 in order to allow a surgical operator to cut the graft 150 without physical interference from the center top set screw 128.

In an aspect, the base insert 130 may further include a removable bottom 112 having a half circle shape, the removable bottom 112 being shown in a half circle shape and removed in FIG. 8. According to this aspect, part of the axial opening 122 persists through the entire cutting jig 100 when the removable bottom 112 is removed. Removing the removal bottom 112 increases the open space of the axial opening 122 in order to allow a surgical operator to cut the graft 150 without physical interference from the removable bottom 112 and further allows for removal of bone fragments cut from the graft 150 which can fall through the axial opening 122 and away from the cutting jig 100.

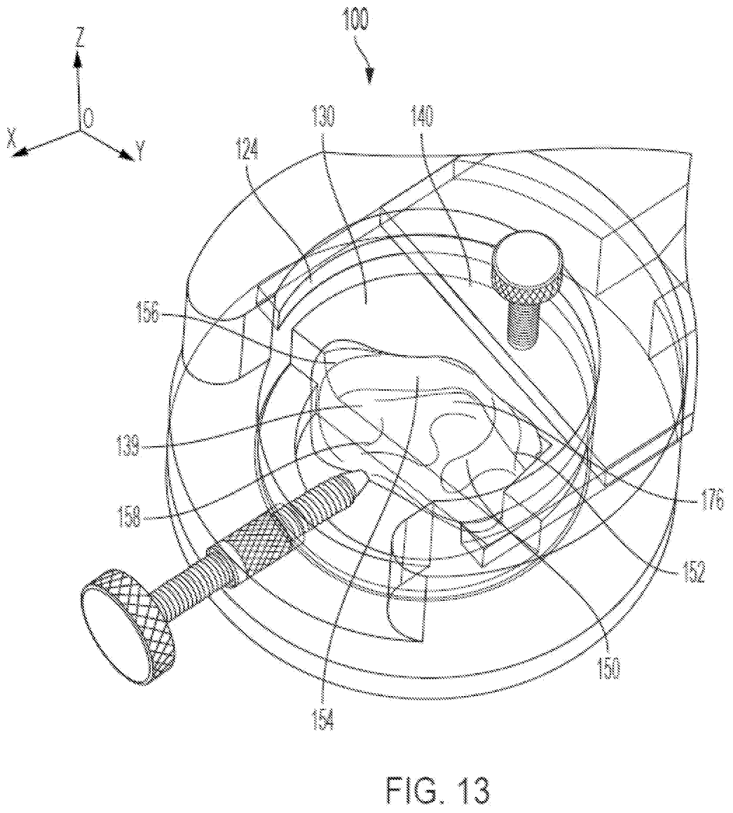

The removable bottom 112 may also be flippable between two configurations within the base insert 130. Any one or more of the removable bottom 112, the graft-receiving area 131, the base insert 130, or portions thereof may be flippable individually or together. In such configurations, the base insert 130 includes on one side a distally-engaging surface 138 configured to match the distal face 176 of the graft 150. Further in such configurations, the base insert 130 includes on the opposing side a proximally-engaging surface 139 configured to match the proximal portion 162 of the graft 150. In one configuration, the distally-engaging surface 138 is oriented to contact the graft 150. In the other configuration, the proximally-engaging surface 139 is oriented to contact the graft 150. The removable bottom 112, the graft-receiving area 131, the base insert 130, or any portion thereof may be flipped from one configuration to the other in order to engage the graft 150 at the distal face 176 or the proximal portion 162. According to one aspect, as shown in FIGS. 6 and 13, a portion of the graft-receiving area 131 is flippable and includes a proximally-engaging surface 139 on one side. Further according to this aspect, as shown in FIGS. 11 and 12, the opposing side includes a distally-engaging surface 138. In this aspect, only the portion of the graft-receiving area 131 including the distally-engaging surface 138 on one side and the proximally-engaging surface 139 is flippable. That is, the graft-engaging surface 136 remains in its relative position before and after flipping the portion of the graft-receiving area 131. In another aspect which is not illustrated, the entire graft-receiving area 131 may be flippable including the graft-engaging surface 136. According to this aspect, a first graft-engaging surface 136 is included on one side, adjacent to the distally-engaging surface 138, and a second graft-engaging surface 136 is included on the opposing side, adjacent to the proximally-engaging surface 139. Additional flippable configurations of the base insert 130 are contemplated, though not illustrated. For example, the entire base insert 130 may be flippable and include a distally-engaging surface 138 on one side and a proximally-engaging surface 139 on the opposing side.

In an aspect and as shown in FIG. 8, the top component 120 may further include a groove 125 and a slidable member 123 containing the slot 124 and the cutting guide 140. The slidable member 123 is positioned within the groove 125 such that the slidable member 123 and cutting guide 140 are together rotatable circumferentially about the top component 120 without physical interference from any of the center top set screw 128 and the outer top set screws 127. Once the slidable member 123 and the cutting guide 140 are positioned appropriately, the second set screw 144 is adjusted to secure the slidable member 123 and the cutting guide 140 to the top component

In an aspect, as shown in FIG. 10, the graft-receiving area 131 is shaped to contact the distal radius bone graft 150 at a scaphoid facet 156 and at a lunate facet 152 of the graft 150 to increase stability and alignment of the graft 150 relative to the cutting jig 100. Specifically, in this example, when the graft 150 is set and secured in the graft-receiving area 131, the graft-engaging surface 136 of the graft-receiving area 131 contacts each of the lunate facet 152, the first surface 154, and the scaphoid facet 156 of the graft 150. In an aspect, and as shown in FIG. 10, the first surface 154 is a dorsal surface 154 and the graft-engaging surface 136 is a dorsal-engaging surface 136 configured to engage the dorsal surface 154. In another aspect, not shown in FIG. 10, the first surface 154 is a palmar surface 154 and the graft-engaging surface 136 is a palmar-engaging surface 136 configured to engage the palmar surface 154. A patient-specific graft-engaging surface 136 can be prepared according to any suitable method, as discussed above with respect to FIG. 6.

FIGS. 10-14 illustrate various stages of an aspect of a method of preparing a distal radius bone graft 150. The method can be performed partially or fully using the cutting jig 100 described above. The distal radius bone graft 150 is obtained and is positioned into the cutting jig 100 such that the first surface 154 or first portion 154 of the graft 150 is removably coupled to the graft-engaging surface 136 of the cutting jig 100 and at least part of a proximal portion 162 and at least part of a portion or surface 158, which is opposite the first surface 154 of the graft 150, are accessible to a surgical operator using the cutting jig 100, as shown. According to an aspect illustrated in FIG. 10, the first surface or portion 154 is a dorsal surface or portion 154 and the graft-engaging surface 136 is a dorsal-engaging surface 136. In another aspect, not shown, the first surface or portion 154 is a palmar surface or portion 154 and the graft-engaging surface 136 is a palmar-engaging surface 136. Throughout this disclosure, “positioning” the graft 150 within the jig 100 can include adjusting the top set screw 128 to contact the graft 150 and to limit or prevent rotation of the top component 120 about the base component 110 as shown and described above with respect to FIG. 1.

A rough depth cut can optionally be performed to form a rough proximal face 164 of the graft 150, shown in FIG. 10, which shows the graft 150 after the rough depth cut has been performed. The rough depth cut is performed on at least part of the proximal portion 162 of the graft 150 to separate the graft 150 from the remainder of the distal radius and to prepare a rough proximal face 164. As shown in FIG. 10, the rough depth cut is performed in a direction approximately normal to the z-axis of the graft 150, the z-axis being approximately normal to the proximal portion 162 and approximately normal to the rough proximal face 164. The rough depth cut can be performed at a separate time or location. That is, the rough depth cut can be performed before the surgical operator obtains the distal radius bone graft 150 as described herein. For instance, the rough depth cut may be performed at a cadaver bone supplier, or may be performed either at the supplier or by the operator to obtain a graft that is roughly the size needed and is ready for a final depth cut. Before cutting, the graft 150 is adjusted such that the proximal portion 162 is aligned relative to the slot 124. Preferably, the graft 150 is adjusted such that the plane of the rough proximal face 164 is aligned to the height of the slot 124.

Once the graft 150 is positioned, the cutting guide 140 is removed from the slot 124 and a resection tool such as a saw is inserted into or passed through the slot 124 and used to cut the graft 150 along the plane of the rough proximal face 164. Alternatively or in conjunction, the resection tool is moved approximately along the recessed portion 126 of the top component 120 in order to cut the graft 150 at a lower height than if the saw were passed through slot 124. The rough depth cut can be performed with aid of the cutting jig 100 as shown, or alternatively can be performed without use of the cutting jig 100. Furthermore, the method of preparing the graft 150 can be carried out without performance of the rough depth cut. While the rough depth cut is performed (if at all) before the final depth cut (discussed below with respect to FIG. 12), it can otherwise be performed before or after any other cutting steps as described herein, specifically the width cut(s) discussed below.

FIG. 11 shows the graft 150 after performance of a rough width cut which formed a rough face 166. In a particular example and as shown in FIG. 11, the rough face 166 is a rough palmar face 166. In another example not shown in FIG. 11, the rough face 166 is a rough dorsal face 166. The rough width cut is performed on at least part of the portion or surface 158, which is opposite the first surface 154 of the graft 150. In the example illustrated in FIG. 11, opposite surface 158 is the palmar face 158 which is prepared to at least remove a cornice 168 of bone on the palmar portion 158 (cornice 168 shown intact with the graft 150 in FIG. 10). In an alternative example, not shown, the opposite surface 158 to be cut is the dorsal surface 158. The rough width cut is performed approximately normal to the x-axis of the graft 150. The x-axis is approximately normal to the plane of the palmar surface 158 and approximately normal to the rough palmar face 166 in the example illustrated in FIG. 11.

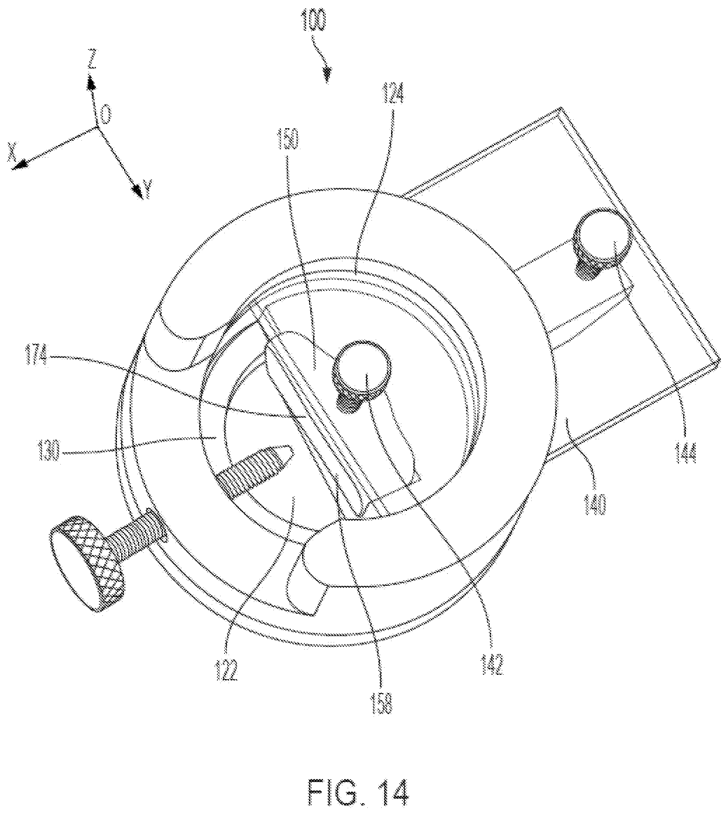

The graft 150 can optionally be aligned to one or more components of the cutting jig 100 and secured in the cutting jig 100 before carrying out the rough width cut in order to perform the cut accurately. In an aspect, the method includes, before performing the rough width cut, aligning the portion 158 (opposite the first surface 154) relative to axial opening 122, the cutting guide 140 (not shown in FIG. 11), and the base insert 130. In an aspect, the graft 150 is positioned such that the rough face 166 is aligned relative to the base insert 130. In an aspect, the graft 150 is positioned such that the rough face 166 is aligned relative to the cutting guide 140. In an aspect, the method further includes engaging the first set screw 142 (not shown in FIG. 11) and if present second set screw 144 (not shown in FIG. 11) to prevent movement of the cutting guide 140 relative to the graft 150. In an aspect, the method further includes passing a resection tool through the axial opening 122 relative to the cutting guide 140 in order to cut at least part of the graft 150 to prepare the rough face 166. Furthermore, the cutting guide 140 may be partially retracted, or fully removed from the cutting jig 100 as shown in FIG. 11, before carrying out the rough width cut. The method of preparing the graft 150 can alternatively be carried out without performance of the rough width cut. The rough width cut is performed (if at all) before the final width cut (discussed below with respect to FIG. 14), but can otherwise be performed before or after any other cutting steps as described herein, such as the depth cut(s) discussed above and below. Furthermore, the rough width cut can be performed at a separate time or location. That is, the rough width cut can be performed before the surgical operator obtains the distal radius bone graft 150 as described herein. Furthermore, the graft 150 having the rough face 166 may be assessed by measuring the graft 150 if the desired width is known, by conducting a trial fit assessment in an open surgical procedure, or by any other suitable assessment methods known in the art. Furthermore, the rough width cut may be performed multiple times as needed to prepare the graft 150 having the desired width.

FIG. 12 shows the graft 150 after performance of a final depth cut to form a prepared proximal face 172. The final depth cut is performed on the proximal portion 162 of the graft 150 to form the prepared proximal face 172. As shown in FIG. 12, the final depth cut is performed in a direction approximately normal to the z-axis of the graft 150, the z-axis being approximately normal to the proximal portion 162 and approximately normal to the prepared proximal face 172. Before cutting, the graft 150 is adjusted such that the proximal portion 162 is aligned relative to the slot 124. Preferably, to specify the location of the final depth cut, a plane of the proximal portion 162 is aligned relative to the height of the slot 124 such that the prepared proximal face 172 is formed when the graft 150 is cut along the plane. That is, the graft 150 is adjusted such that the prepared proximal face 172, once formed by the final depth cut, is aligned to the height of the slot 124. Once the graft 150 is positioned as desired by a surgical operator, the cutting guide 140 (not shown in FIG. 12) is removed from the slot 124 and a resection tool such as a saw is inserted into or passed through the slot 124 and used to cut the graft 150 along the plane of the proximal portion 162 (i.e., normal to the z-axis as shown in FIG. 12) to form the prepared proximal face 172. Alternatively, the resection tool is moved approximately along the recessed portion 126 of the top component 120 in order to cut the graft 150. The final depth cut is performed with aid of the cutting jig 100 as shown. The final depth cut is performed after the optional rough depth cut, but can otherwise be performed before or after any other cutting steps as described herein, such as the width cut(s) discussed herein.

FIG. 13 shows the graft 150 after the graft-receiving area 131 of the base insert 130 has been flipped or rotated. Flipping the graft-receiving area 131 reverses the positions of the lunate facet 152 and the scaphoid facet 156. Comparing with FIG. 10, for instance, it can be seen that the lunate facet 152 has moved from a leftward position to a rightward position, and the scaphoid facet 156 has moved from a rightward position to a leftward position, and furthermore, each of the lunate facet 152 and the scaphoid facet 156 face upward after flipping. According to the example illustrated in FIG. 13, after flipping, the dorsal surface 154 remains in its relative position, though now inverted in relation to the orientation shown in FIG. 10. The orientation of the graft 150 in FIG. 10 is to be understood herein as an “upside down” orientation, whereas the orientation of the graft 150 in FIG. 13 is to be understood herein as a “right side up” orientation.

After flipping, the graft 150 contacts the graft-engaging surface 136 and the proximally-engaging surface 139, as shown in FIG. 13. By contrast, FIGS. 11 and 12 show the graft 150 contacting the distally-engaging surface 138 before flipping. As discussed above with respect to FIG. 6, each of the distally-engaging surface 138 and the proximally-engaging surface 139 may independently be shaped to match one or more surfaces of the graft 150. Surfaces 138 and 139 may each independently be patient-specific or have a generic shape. Surfaces 138 and 139 may each independently be determined by measuring dimensions and curvatures of one or more surfaces of the donor's distal radius, measuring dimensions and curvatures of one or more surfaces of the recipient patient's glenoid, preparing an average surface of the donor's distal radius by analyzing multiple distal radii in a lookup table or a database, or other suitable methods known in the art, and surfaces 138 and 139 may each independently be prepared by 3D printing or other suitable methods known in the art. In an aspect, the proximally-engaging surface 139 matches a common surface of a plurality of grafts. In an aspect, the base insert 130 is formed using a 3D printing process such that the proximally-engaging surface 139 matches the three-dimensional shape of the graft 150. In an aspect, the distally-engaging surface 138 matches a common surface of a plurality of grafts. In an aspect, the base insert 130 is formed using a 3D printing process such that the distally-engaging surface 138 matches the three-dimensional shape of the graft 150.

Flipping the graft-receiving area 131 gives a surgical operator a more accessible view to plan and perform the final width cut, the final width cut being discussed below with respect to FIG. 14. Specifically, and as shown in FIG. 13, flipping the graft-receiving area 131 causes the distal face 176 to face upward toward the surgical operator and causes each of the lunate facet 152 and the scaphoid facet 156 to face upward toward the surgical operator, which allows the surgical operator to see the lunate facet 152 and the scaphoid facet 156 before cutting and determine how much of the lunate facet 152 and the scaphoid facet 156 to remove in the final width cut. Without flipping the graft 150 right side up, the surgical operator would be forced to begin cutting at the opposite end of the palmar surface 158 with the lunate facet 152 and the scaphoid facet 156 facing downward and away from the surgical operator (as shown in FIG. 10, for instance), and the surgical operator would have to guess or approximate the best location to begin the cut. In an aspect, the method further includes repositioning the base insert 130 to expose the distal face 176 of the graft 150. In another aspect, the repositioning is carried out by flipping the graft-receiving area 131 of the base insert 130 to expose the distal face 176 of the graft 150 to a surgical operator. In a further aspect, the repositioning is carried out after performance of the rough width cut.

FIG. 14 shows the graft 150 after a final width cut has been performed to form a prepared palmar face 174. The final width cut is performed on the palmar portion 158 of the graft 150 (the palmar portion 158 being shown more fully before being cut in FIG. 13). As shown in FIG. 14, the final width cut is performed in a direction approximately normal to the x-axis of the graft 150, the x-axis being approximately normal to the plane of the palmar surface 158 and approximately normal to the prepared palmar face 174. According to the example shown in FIG. 14, the portion or surface 158, which is opposite the first surface 154, may be a palmar portion 158 or palmar surface 158, and the prepared face 174 may be a prepared palmar face 174. Or in another example, not shown in FIG. 14, the portion or surface 158 opposite the first surface 154 may be a dorsal portion 158 or dorsal surface 158, and the prepared face 174 may be a prepared dorsal face 174.

The graft 150 is aligned to one or more components of the cutting jig 100 and secured in the cutting jig 100 before carrying out the final width cut in order to perform the cut accurately. The cutting guide 140 can be used as a physical guide to partially restrict radial movement of the resection tool during cutting and allow full axial movement of the resection tool. In an aspect, the method includes, before performing the final width cut, aligning the portion 158 (opposite the first surface 154) relative to at least one of the slot 124, the cutting guide 140, the base insert 130, or the axial opening 122. In an aspect, the graft 150 is positioned such that the prepared face 174 is aligned relative to the base insert 130 as shown in FIG. 14. In an aspect, the graft 150 is positioned such that the prepared face 174 is aligned relative to the cutting guide 140 as shown in FIG. 14. In an aspect, the method further includes engaging either or both of the first set screw 142 and the second set screw 144 to prevent movement of the cutting guide 140 relative to the graft 150. In an aspect, the method further includes passing a resection tool through the axial opening 122 relative to the cutting guide 140 in order to cut at least part of the graft 150 to form the prepared face 174. The final width cut is performed after the optional rough width cut, but can otherwise be performed before or after any other cutting steps as described herein, such as the depth cut(s) discussed above. In an aspect, the final width cut is performed after the final depth cut described above with reference to FIG. 12. In an aspect, the final depth cut and the final width cut are each performed in a direction approximately perpendicular to each other.

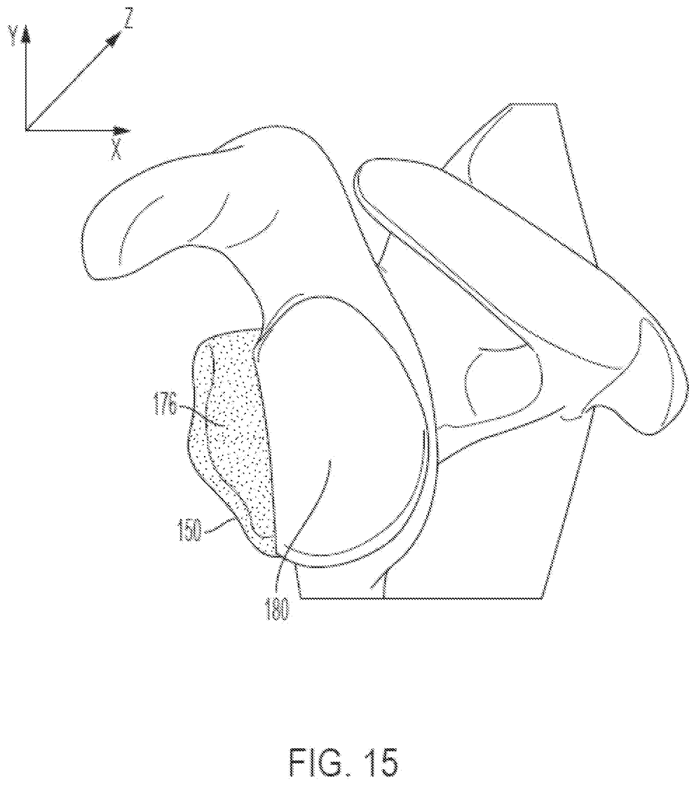

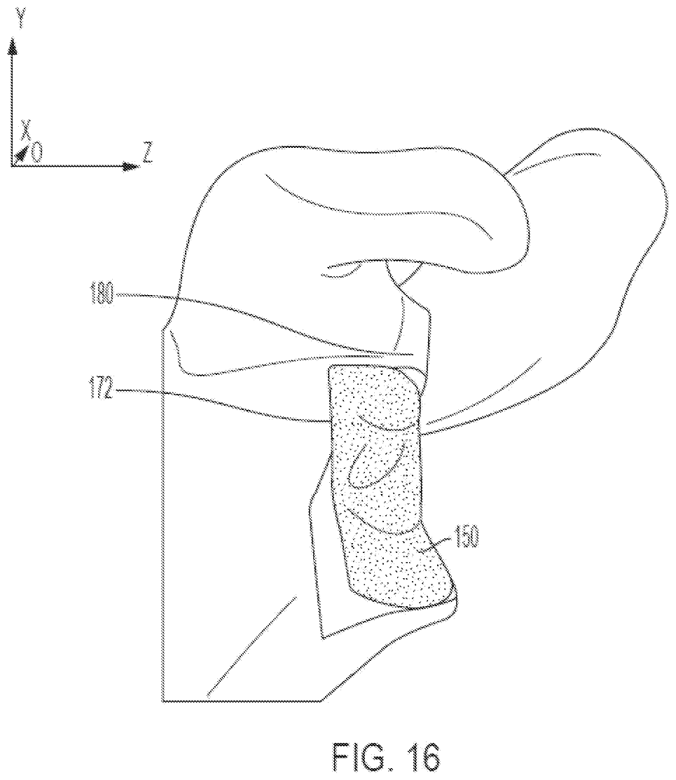

FIGS. 15 and 16 show a prepared graft 150 aligned to a patient glenoid 180. FIG. 15 shows the distal face 176 when the exemplary prepared palmar face interacts with the prepared surface of the glenoid defect, and FIG. 16 shows the prepared proximal face 172 after execution of the final depth cut. The graft 150 may be prepared to fit neatly to the patient glenoid 180. As discussed above with respect to FIG. 6, such fitting of the graft 150 to one or more glenoid surfaces of a patient glenoid 180, as well as determinations of the locations for the width and depth cuts, may be made by any suitable measurement methods known in the art, such as a CT scan. Furthermore, as discussed above with respect to FIG. 6, such determinations of average curvatures and dimensions of one or more glenoid surfaces for each of a plurality of glenoids, or of one or more distal radius surfaces for each of a plurality of distal radii, may be made by any suitable measurement methods known in the art, such as a lookup table or a database such as SOMA. In an aspect, prior to performance of each of the final width cut and the final depth cut, the method includes determining curvatures and dimensions of one or more surfaces of a patient glenoid 180 such that following performance of each of the final width cut and the final depth cut, curvatures and dimensions of the prepared proximal face 172 and the prepared face 174 match the curvatures and dimensions of the one or more surfaces of the patient glenoid 180. In an aspect, prior to performance of each of the final width cut and the final depth cut, the method includes determining average curvatures and dimensions of one or more glenoid surfaces for each of a plurality of glenoids from a database, and further includes performing the final width cut and the final depth cut such that the curvatures and dimensions of the prepared proximal face 172 and the prepared face 174 match the average curvatures and dimensions of the one or more glenoid surfaces.

After fitting the prepared graft 150 to the patient glenoid 180, the fit may be analyzed by measuring the graft 150, performing a trial fit assessment in an open surgical procedure, performing follow-up assessments with the patient after specified periods of time, for example, daily or weekly, or by any suitable assessment methods known in the art or combinations thereof. In an aspect, the method may further include assessing the fit of the distal radius bone graft 150 relative to the glenoid 180 and repeating the steps of positioning the graft 150 in the cutting jig 100, and performing the final depth cut and/or performing the final width cut as necessary to revise the fit.

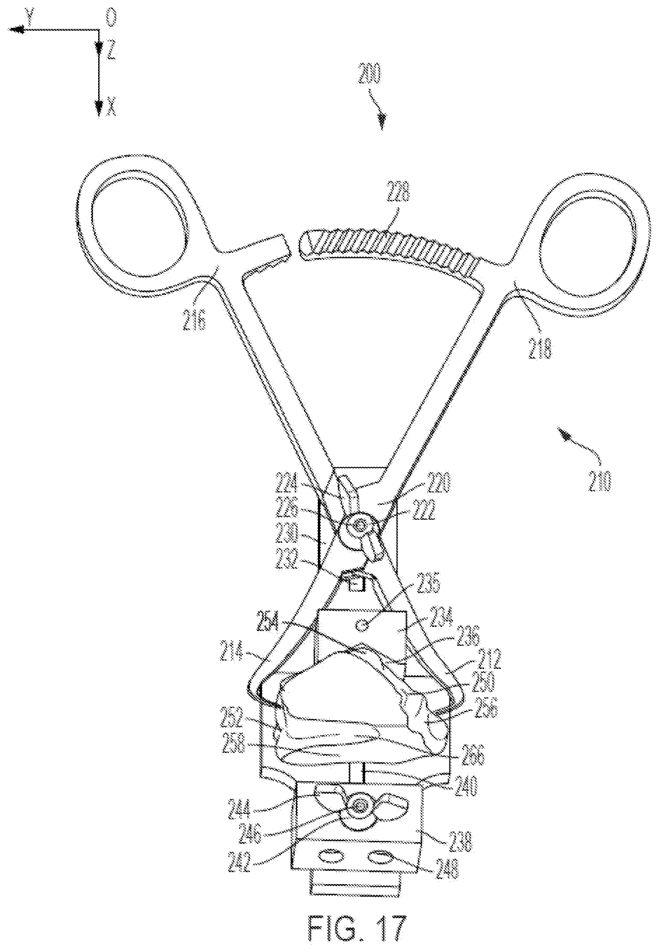

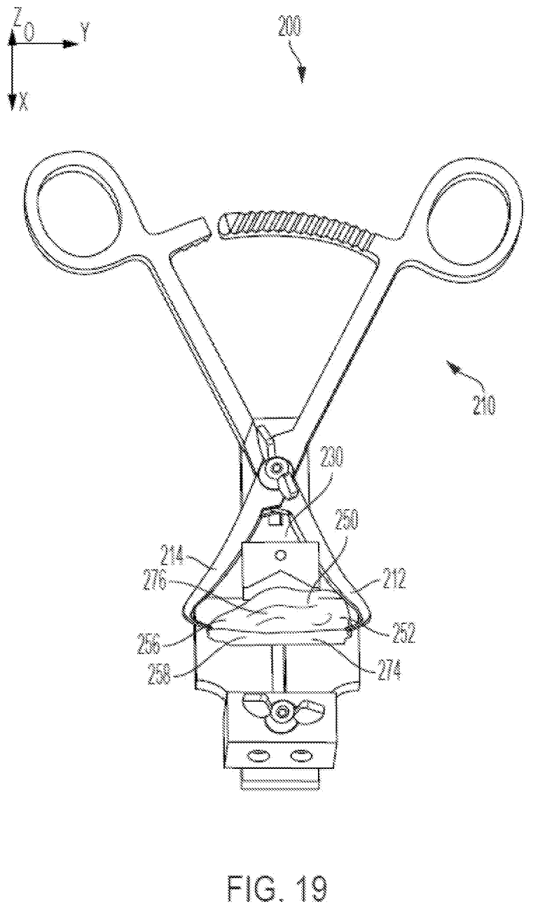

FIGS. 17-22 illustrate a clamp 200 according to an exemplary aspect and further illustrate various stages of an aspect of a method of preparing the distal radius bone graft 250. As shown in FIG. 17, the clamp 200 is provided for securing and manipulating the graft 250. The clamp includes a securing tool 210 for securing the graft 250 within the clamp 200. The securing tool 210 includes a first securement feature 212 and a second securement feature 214, each of which are configured to contact and secure the graft 250. Such securement features 212 and 214 may include first and second prongs 212 and 214 as shown in FIG. 17, teeth 212 and 214, ridges 212 and 214, first and second specialized geometries 212 and 214 configured to optimize securement of a given graft feature, or the like. In a first configuration, the first securement feature 212 contacts the graft 250 at the scaphoid facet 256 and the second securement feature 214 contacts the graft 250 at the lunate facet 252. In a second configuration, as shown in FIG. 19 and discussed below, the first securement feature 212 contacts the lunate facet 252 and the second securement feature 214 contacts the scaphoid facet 256. In an aspect, the first securement feature 212 and the second securement feature 214 are each a single securement feature. In another aspect, the first securement feature 212 and the second securement feature 214 each independently include one or more securement features. For instance, the first securement feature 212 may include a plurality of first prongs 212, each contacting the graft 250 at the scaphoid facet 256. Such a plurality of first securement features 212 or second securement features 214 allows for a more uniform distribution of pressure on the graft 250, ensuring stability in the clamp 200. The first securement feature 212 is connected to a first end 216 and the second securement feature 214 is connected to a second end 218.

The fulcrum housing 220 includes a fulcrum connector 222 to connect the first securement feature 212 and first end 216 to the second securement feature 214 and second end 218, and as shown in FIG. 17, the fulcrum connector 222 may include a fulcrum wing nut 224 and a fulcrum screw 226. The fulcrum housing 220 is configured to allow for relative horizontal (i.e., along the x-axis as shown in FIG. 17) movement of the first securement feature 212, second securement feature 214, first end 216, and second end 218 such that when the first end 216 and second end 218 are moved toward one another by a surgical operator, the first securement feature 212 and second securement feature 214 are moved toward one another to contact and secure the graft 250, and vice versa.

Furthermore, the securing tool 210 may include a ratchet 228. In an aspect, and as shown in FIG. 17, the ratchet 228 may include two separate pieces having ridges on opposing sides, as in opposing ratchet teeth. Once a surgical operator sets the first end 216 and second end 218 (and thus the first securement feature 212 and second securement feature 214) at a certain distance from one another, the opposing ridges of the ratchet 228 can be configured to interlock and thus maintain that set distance without further physical input from the surgical operator. Thus the first securement feature 212, second securement feature 214, first end 216, second 218, and the ratchet 228 may be together configured to continuously secure the graft 250 without continuous or repeated physical input from the surgical operator.

The clamp 200 further includes a base 230 having a fulcrum channel 232. The fulcrum channel 232 is configured to connect the base 230 to the securing tool 210. More specifically, the fulcrum channel 232 is configured to allow the fulcrum connector 222 to connect the base 230 to the securing tool 210. In a particular aspect, and as shown in FIG. 17, the fulcrum screw 226 is located within the fulcrum housing 220 and the fulcrum channel 232, and the fulcrum wing nut 224 is adjustable to press the fulcrum housing 220 and the base 230 toward one another and tighten and maintain the connection therebetween. Furthermore, the fulcrum channel 232 is oriented such that the securing tool 210 can be moved toward or away from the graft 250 and/or graft-engaging block 234 by moving along the channel 232. The fulcrum housing 220 is adjustable to loosen or tighten the connection between the fulcrum housing 220 and the base 230, such that a surgical operator can loosen the fulcrum housing 220 to allow for movement of the securing tool 210 within the channel 232, and can then tighten the fulcrum housing 220 to secure the securing tool 210 at a certain position relative to the base 230, which may allow for some flexibility into the clamp 200 system, such as flexibility in positioning graft 250 relative to the base 230, flexibility in allowing for various shaped and sized grafts 250, or the like. In a particular aspect and as shown in FIG. 17, the fulcrum wing nut 224 is adjustable to perform such tightening and loosening of the fulcrum housing 220.

The base 230 further includes a graft-engaging block 234 which includes a graft-engaging surface 236. The graft-engaging surface 236 may be similar to the above-described graft-engaging surface 136. In an aspect and as shown in FIG. 17, the graft-engaging surface 236 includes a V-block shape (as mentioned above) configured to contact and secure the first surface 254 of the graft 250. In another aspect, not shown in FIG. 17 but similar to what is discussed above with respect to FIG. 6, the graft-engaging surface 236 is configured to match the three-dimensional shape of the first surface 254 of the graft 250. In a further aspect, not shown in FIG. 17 but similar to what is discussed above with respect to FIG. 6, the graft-engaging surface 236 is formed using a 3D printing process such that the graft-engaging surface 236 matches the three-dimensional shape of the first surface 254. The first surface or portion 254 may be a dorsal surface or portion 254 and the graft-engaging surface 236 may be a dorsal-engaging surface 236. Or, the first surface or portion 254 may be a palmar surface or portion 254 and the graft-engaging surface 236 may be a palmar-engaging surface 236. Further, similar to what is described above with respect to FIG. 6, though not shown, the graft-engaging surface 236 may include a plurality of modular inserts configured to match the average curvatures and dimensions of one or more graft surfaces. Specifically, the plurality of modular inserts may be configured to match the average curvatures and dimensions of any one or more of the first surface 254, the surface 258 which is opposite the first surface 254, the lunate facet 252, the scaphoid facet 256, the proximal portion 262, or the distal face 276. The first securement feature 212, second securement feature 214, base 230, and graft-engaging block 234 together contact and secure the graft 250 within the clamp 200.

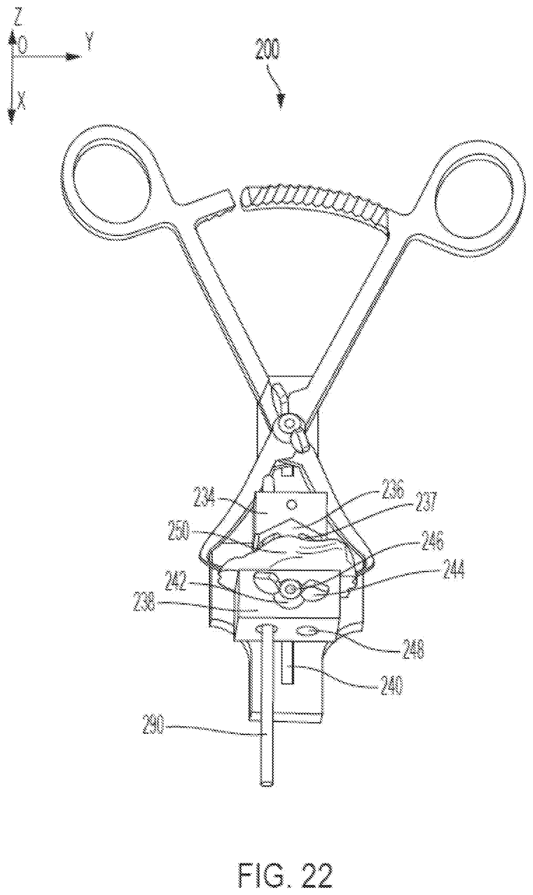

As shown in FIG. 17, the graft-engaging block 234 may further include a throughhole 235 extending through the base 230 and through a guide-engaging channel 240, which allows for the graft-engaging block 234 to connect with and be secured to the base 230 when a connecting member such as a set screw (not shown in FIG. 17) is inserted into the throughhole 235. In another aspect, the set screw (not shown in FIG. 17) can instead be a cross pin (not shown in FIG. 17) inserted into the throughhole 235. The block 234 could therefore rotate around hole 235 if desired to allow a surgical operator to find an optimized stable location for the graft 250 while the securement features 212 and 214 are being applied. The base 230 further includes a drilling guide 238 and the guide-engaging channel 240. The drilling guide 238 includes a guide connector 242. The guide connector 242 and the guide-engaging channel 240 are similar to the fulcrum connector 222 and the fulcrum channel 232 in that the guide-engaging channel 240 is configured to allow the guide connector 242 to connect the base 230 to the drilling guide 238 and the guide-engaging channel 240 is oriented such that the drilling guide 238 can be moved toward or away from the graft 250 by moving along the channel 240. In a particular aspect and as shown in FIG. 17, the guide connector 242 includes a guide wing nut 244 and a guide screw 246. The guide wing nut 244 and the guide screw 246 are similar to the fulcrum wing nut 224 and the fulcrum screw 226 in that the guide screw 246 is located within the drilling guide 238 and the guide-engaging channel 240, and the guide wing nut 244 is adjustable to press the drilling guide 238 toward the base 230 and tighten the connection therebetween. The drilling guide 238 further includes a plurality of guide channels 248 and the drilling guide 238 is configured to drill a plurality of channels through the graft 250 when a drill bit is passed through the guide channels 248, which is described in detail below with respect to FIG. 22.

The method can be performed partially or fully using the clamp 200 described above. The distal radius bone graft 250 is obtained and is positioned into the clamp 200 such that the first surface 254 or first portion 254 of the graft 250 is removably coupled to the graft-engaging surface 236 of the clamp 200 and at least part of a proximal portion 262 and at least part of a surface or portion 258 (opposite the first surface 254 of the graft 250) are accessible to a surgical operator using the clamp 200, as shown. Throughout this disclosure, “positioning” the graft 250 within the clamp 200 may include moving the first end 216 and second end 218 toward one another to cause the first securement feature 212 and the second securement feature 214 to contact, engage with, and secure the graft 250 within the clamp 200, and may further include configuring the ratchet 228 to maintain the set distance between the first securement feature 212 and the second securement feature 214, both of which are described above with respect to FIG. 17. Furthermore, “positioning” the graft 250 may further include placing the graft 250 on the base 230 such that the first surface 254 of the graft 250 contacts and secures the graft-engaging surface 236 of the graft-engaging block 234. In an aspect, the method of preparing the graft 250 includes engaging each of the first securement feature 212 and the second securement feature 214 with the graft 250 to secure the graft 250 within the clamp 200.

FIG. 17 shows the graft 250 after a rough width cut has been performed on the graft 250 to form a rough face 266. The rough face 266 may be a rough palmar face 266, as shown in FIG. 17, or, not shown in FIG. 17, may be a rough dorsal face 266. The rough width cut is performed on at least part of the portion or surface 258, which is opposite the first surface 254, of the graft 250 in order to at least remove a cornice of bone on the portion 258 (such cornice being the same as cornice 168, which is shown intact with the graft 150 in FIG. 10). As shown in FIG. 17, the rough width cut is performed in a direction approximately normal to the x-axis of the graft 250, the x-axis being approximately normal to the plane of the surface 258 and approximately normal to the rough face 266. The graft 250 may be secured in the clamp 200 before carrying out the rough width cut, and particularly may be secured against the graft-engaging block 234 before carrying out the rough width cut. The method of preparing the graft 250 can be carried out with or without performance of the rough width cut. The rough width cut is performed (if at all) before the final width cut (discussed below with respect to FIG. 19), but can otherwise be performed before or after any other cutting steps as described herein, such as the depth cut(s) discussed above and below. Furthermore, the rough width cut can be performed at a separate time or location. That is, the rough width cut can be performed before the surgical operator obtains the distal radius bone graft 250 as described herein. Furthermore, the graft 250 having the rough face 266 may be assessed by measuring the graft 250 if the desired width is known, by conducting a trial fit assessment in an open surgical procedure, or by any other suitable assessment methods known in the art. Furthermore, the rough width cut may be performed multiple times as needed to prepare the graft 250 having the desired width.

As shown in FIG. 17, the graft 250 is secured in the clamp 200 via contact between the graft 250 and the base 230, contact between the first securement feature 212 and the scaphoid facet 256, contact between the second securement feature 214 and the lunate facet 252, and contact between the first surface 254 and the graft-engaging surface 236, all of which are adjustable to one another to accommodate operator preferences, different sized and shaped grafts, and the like. The surface 258 (which is opposite the first surface 254) does not contact the clamp 200, the first securement feature 212, nor the second securement feature 214, and thus the surface 258 is not relied on to secure the graft 250. This allows the surface 258 to be cut (and the rough face 266 to be formed) without altering any other contact points between the graft 250 and the clamp 200. Stated another way, the rough width cut can be performed while the clamp 200, and particularly the securing tool 210, continuously contacts and secures the graft 250, without need for repositioning and resecuring the graft 250 during performance of the cut. In an aspect, the securing tool 210 is configured to continuously contact and secure the graft 250 while at least part of the portion 258 is removed from the graft 250. More specifically, the securing tool 210 may be configured to continuously contact and secure the graft 250, without contacting the portion 258, while the rough width cut, the final width cut, or both are performed. Even more specifically, the first securement feature 212 and the second securement feature 214 may be configured to continuously contact and secure the graft 250, without contacting the portion 258, and by contacting the graft 250 at positions such that the rough width cut, the final width cut, or both may be performed without physical interference from the first securement feature 212 and the second securement feature 214.

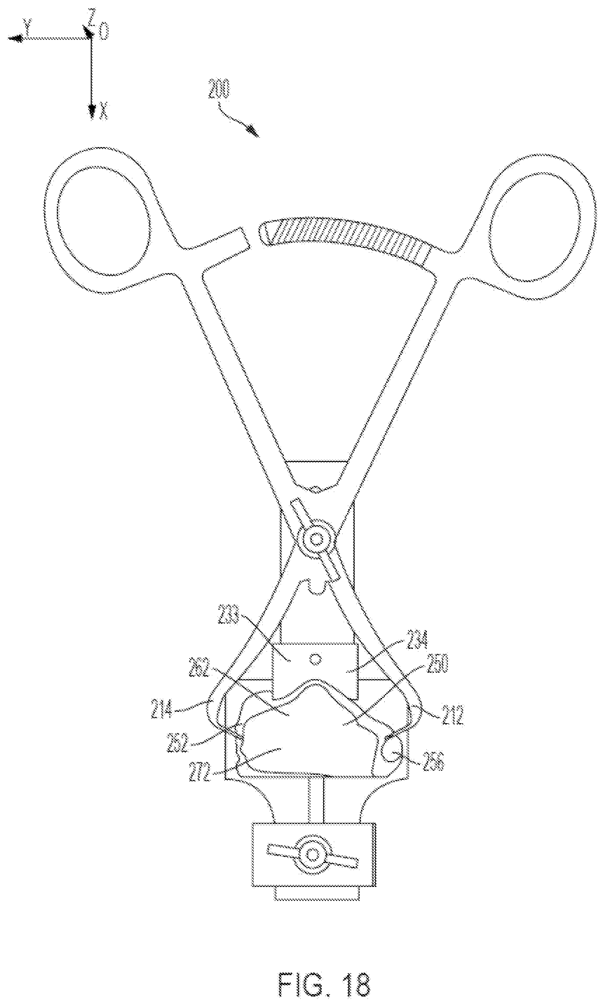

FIG. 18 shows the graft 250 after a final depth cut has been performed. The final depth cut is performed on the proximal portion 262 of the graft 250 to form the prepared proximal face 272. As shown in FIG. 18, the final depth cut is performed in a direction approximately normal to the z-axis of the graft 250, the z-axis being approximately normal to the proximal portion 262 and approximately normal to the prepared proximal face 272. Once the graft 250 is positioned as desired by a surgical operator, the graft 250 is cut along the plane of the proximal portion 262 (i.e., normal to the z-axis as shown in FIG. 18) to form the prepared proximal face 272. The final depth cut is performed with aid of the clamp 200 as shown. Particularly, a top surface 233 of the graft-engaging block 234 may be used to assist performance of the final depth cut, like the cutting guide 140 of the cutting jig 100 described above. Similar to what is described above with reference to FIG. 12, before cutting, the graft 250 is positioned such that the proximal portion 262 is aligned relative to the height of the top surface 233. That is, the graft 250 is adjusted such that the prepared proximal face 272, once formed by the final depth cut, is aligned to the height of the top surface 233. Once the graft 250 is positioned as desired by a surgical operator, a resection tool such as a saw is passed along the top surface 233 to cut the graft 250 along the plane of the proximal portion 262 (i.e., normal to the z-axis as shown in FIG. 18) to form the prepared proximal face 272. The final depth cut is performed after the optional rough depth cut described below, but can otherwise be performed before or after any other cutting steps as described herein, such as the width cut(s) discussed herein.

Although not shown in FIG. 18, a rough depth cut may be performed on the graft 250 before the final depth cut is performed, as similarly described above with respect to the cutting jig 100 in FIG. 10. That is, the rough depth cut can optionally be performed to form a rough proximal face of the graft 250. The rough depth cut is performed on at least part of the proximal portion 262 of the graft 250 to separate the graft 250 from the remainder of the distal radius and to prepare a rough proximal face. Like the final depth cut, the rough depth cut is performed in a direction approximately normal to the z-axis of the graft 250, the z-axis being approximately normal to the proximal portion 262 and approximately normal to the rough proximal face. The rough depth cut can be performed at a separate time or location. That is, the rough depth cut can be performed before the surgical operator obtains the distal radius bone graft 250 as described herein. For instance, the rough depth cut may be performed at a cadaver bone supplier, or may be performed cither at the supplier or by the operator to obtain a graft that is roughly the size needed and is ready for a final depth cut. The rough depth cut can be performed with aid of the clamp 200, or alternatively can be performed without use of the clamp 200. Furthermore, the method of preparing the graft 250 can be carried out without performance of the rough depth cut. While the rough depth cut is performed (if at all) before the final depth cut, it can otherwise be performed before or after any other cutting steps as described herein, specifically the width cut(s) discussed below.

FIG. 19 shows the graft 250 after being flipped or rotated and after a final width cut has been performed on the graft 250. The graft 250 may be flipped or rotated by disengaging the first securement feature 212 and second securement feature 214 from the graft 250, then physically removing the graft 250 from the clamp 200, then flipping or rotating the graft 250, and then repositioning the flipped or rotated graft 250 within the clamp 200. Such repositioning may include engaging the first securement feature 212 and the second securement feature 214 to contact the graft 250 as described above with respect to FIG. 17. Flipping the graft 250 reverses the position of the lunate facet 252 and the scaphoid facet 256. Comparing with FIG. 18, for instance, it can be seen in FIG. 19 that the lunate facet 252 has moved from a leftward position to a rightward position, and the scaphoid facet 256 has moved from a rightward position to a leftward position, and furthermore, each of the lunate facet 252 and the scaphoid facet 256 face upward after flipping. After flipping, the first surface 254 remains in its relative position, though now inverted in relation to the orientation shown in FIG. 18. The orientation of the graft 250 in FIG. 18 is to be understood herein as an “upside down” orientation, whereas the orientation of the graft 250 in FIG. 19 is to be understood herein as a “right side up” orientation.

Flipping or rotating the graft 250 to the orientation shown in FIG. 19 gives a surgical operator a more accessible view to plan and perform the final width cut, as described above with respect to the cutting jig 100 in FIG. 13. Specifically, flipping the graft 250 causes the distal face 276 to face upward toward the surgical operator and causes each of the lunate facet 252 and the scaphoid facet 256 to face upward toward the surgical operator, which allows the surgical operator to see the lunate facet 252 and the scaphoid facet 256 before cutting and determine how much of the lunate facet 252 and the scaphoid facet 256 to remove in the final width cut. Without flipping the graft 250 right side up, the surgical operator would be forced to begin cutting at the opposite end of the surface 258 (surface 258 being opposite the first surface 254) with the lunate facet 252 and the scaphoid facet 256 facing downward and away from the surgical operator (as shown in FIG. 18), and the surgical operator would have to guess or approximate the best location to begin the cut. In an aspect, the method of preparing the graft further includes removing the graft 250 from the clamp 200 and further includes repositioning the graft 250 into the clamp 200 such that the first portion 254 is removably coupled to the graft-engaging surface 236 and such that the graft 250 is flipped to expose the distal face 276 to a surgical operator. In a further aspect, the repositioning is carried out before performance of the final width cut. Specifically, the removing may include disengaging the first securement feature 212 and the second securement feature 214 from the graft 250, and the repositioning may include engaging the first securement feature 212 and the second securement feature 214 with the graft 250 to secure the graft 250 within the clamp 200, as discussed above with respect to FIG. 17.

FIG. 19 furthermore shows the graft 250 after a final width cut has been performed to form a prepared face 274. The prepared face 274 may be a prepared palmar face 274 as shown in FIG. 19, or, not shown in FIG. 19, the prepared face 274 may be a prepared dorsal face 274. The final width cut is performed on the portion 258 of the graft 250 (the portion 258 being shown more intact in FIG. 18 before the final width cut is carried out in FIG. 19). As shown in FIG. 19, the final width cut is performed in a direction approximately normal to the x-axis of the graft 250, the x-axis being approximately normal to the plane of the surface 258 and approximately normal to the prepared face 274. The graft 250 is secured in the clamp 200 before carrying out the final width cut in order to perform the cut accurately. The graft 250 may be secured via the first securement feature 212 and the second securement feature 214 as described above with respect to FIG. 17. The final width cut is performed after the optional rough width cut, but can otherwise be performed before or after any other cutting steps as described herein, such as the depth cut(s) discussed above. In an aspect, the final width cut is performed after the final depth cut described above with reference to FIG. 18. In an aspect, the final depth cut and the final width cut are each performed in a direction approximately perpendicular to each other.

As shown in FIG. 19, the graft 250 is secured in the clamp 200 at least via contact between the graft 250 and the base 230, contact between the first securement feature 212 and the lunate facet 252, and contact between the second securement feature 214 and the scaphoid facet 256. Similar to what is described above with respect to FIG. 17, the surface 258 (which is opposite the first surface 254) does not contact the clamp 200 and is not relied on to secure the graft 250. This allows the surface 258 to be cut (and the prepared face 274 to be formed) without altering any other contact points between the graft 250 and the clamp 200. Stated another way, the final width cut can be performed while the clamp 200, and particularly the securing tool 210, continuously contact and secure the graft 250, without need for repositioning and resecuring the graft 250 during performance of the cut. In an aspect, the securing tool 210 is configured to continuously contact and secure the graft 250 while at least part of the portion 258 is removed from the graft 250.

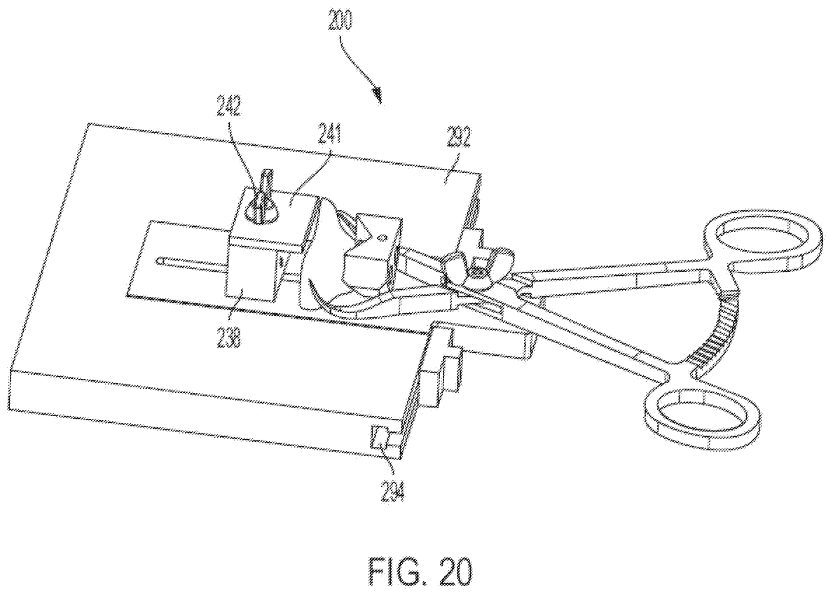

The final width cut may be performed with aid of a cutting guide 241 as shown in FIG. 20, or may be performed freehand without the cutting guide 241. As illustrated, the cutting guide 241 is secured to the drilling guide 238 by the guide connector 242. The cutting guide 241 is rotatable about the drilling guide 238 when the guide connector 242 is loosened, and the cutting guide 241 is secured against the drilling guide 238 when the guide connector 242 is tightened. The cutting guide 241 may be rotated into the position shown in FIG. 20 in order to hang over the drilling guide 238 and physically aid a surgical operator performing the cut, like the cutting guide 140 of the jig 100 and the graft-engaging block 234, discussed above. Further, the cutting guide 241 gives a surgical operator full visibility of the graft 250 while performing the cut. When not in use, the cutting guide 241 can be rotated into a position such that it hangs over the opposite side of the drilling guide 238 and does not interfere with the graft 250 (this position of the cutting guide 241 is not shown). The final width cut and the rough width cut may each be performed independently with or without use of the cutting guide 241. The clamp 200 may further include an optional base 292 as shown in FIG. 20. The optional base 292 includes a base groove 294 which allows the clamp 200 to be secured to another stable structure such as a table, freeing the surgical operator to use both hands when performing the cuts described herein.

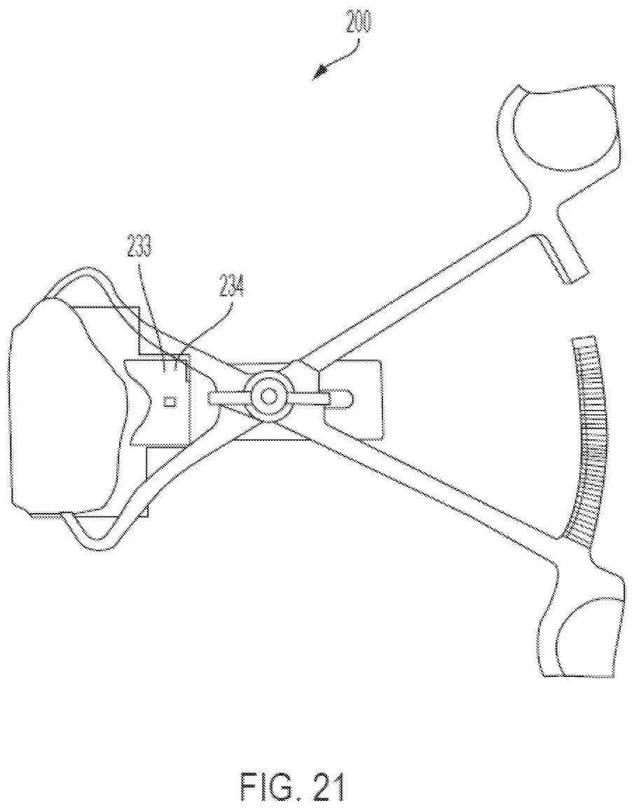

FIG. 21 shows the clamp 200 including a modular guide 296 having an optional rectangular portion 298. As shown, the modular guide 296 is removably secured to the graft-engaging block 234. The modular guide 296 and rectangular portion 298 can function as physical cutting guides, like the cutting guide 140 of the jig 100, the graft-engaging block 234, and the cutting guide 241 discussed above. The modular guide 296 and rectangular portion 298 can each be used as cutting guides for any of the rough or final width and depth cuts described herein. If instead the top surface 233 of the graft-engaging block 234 is used as a cutting guide to perform one or more of the rough depth cut and the final depth cut, the modular guide 296 should be removed prior to cutting in order to allow the top surface 233 of the graft-engaging block 234 to be accessed.

FIG. 22 shows the graft 250 after a plurality of graft channels have been drilled through the graft 250 by a drill bit 290. In one aspect where the block 234 is not allowed to rotate with respect to base 230, the guide channels 248 shall stay approximately aligned with the block channels 237. Starting the drilling from either the guide channels 248 as shown in FIG. 22 or starting the drilling from the block channels 237 could be envisioned. In another aspect where the block 234 is allowed to rotate with respect to the base 230 via a cross pin as previously described, the guide channels 248 may not stay aligned with the block channels 237. In this aspect, the graft interfacing sides of both the block 234 and guide 238 shall potentially include more generous cupping or funneled or chamfered features integrated with the channels such that a drill entering from either direction would be able to drill all the way through the graft and then get temporarily captured or contained on the opposite side. Before drilling, the drilling guide 238 is positioned against the portion 258 (which is opposite the first surface 254 of the graft 250) by sliding the drilling guide 238 along the guide engaging channel 240. Such positioning includes adjusting the guide connector 242 to allow the drilling guide 238 to move along the guide-engaging channel 240, then moving the drilling guide 238 along the guide-engaging channel 240 to contact the portion 258, and then adjusting the guide connector 242 to secure the drilling guide 238 against the graft 250. In a particular aspect, and as shown in FIG. 22, the guide connector 242 includes the guide wing nut 244 and guide screw 246, the adjusting of the guide connector 242 to allow the drilling guide 238 to move includes loosening the guide wing nut 244 and the guide screw 246, and the adjusting of the guide connector 242 to secure the drilling guide 238 includes tightening the guide wing nut 244 and the guide screw 246.

The plurality of graft channels is drilled through the graft 250 after the graft 250 has been prepared to fit to a patient glenoid, that is, after at least the final depth cut and the final width cut are performed. The graft channels can be used to affix the graft 250 to the patient glenoid. For instance, screws can be placed in the graft channels and configured to adjustably secure the graft 250 to the patient glenoid. Furthermore, before or after drilling the graft channels, the graft 250 is aligned to the patient glenoid as described above with respect to FIGS. 15 and 16. The graft 250 may be prepared to fit neatly to the patient glenoid. As discussed above with respect to FIGS. 6, 15, and 16, such fitting of the graft 250 to one or more glenoid surfaces of a patient glenoid, as well as determinations of the locations for the width and depth cuts, may be made by any suitable measurement methods known in the art, such as a CT scan. Furthermore, as discussed above with respect to FIGS. 6, 15, and 16, such determinations of average curvatures and dimensions of one or more glenoid surfaces for each of a plurality of glenoids from a database may be made by any suitable measurement methods known in the art, such as a lookup table or a database such as SOMA. In an aspect, prior to performance of each of the final width cut and the final depth cut, the method includes determining curvatures and dimensions of one or more surfaces of a patient glenoid such that following performance of each of the final width cut and the final depth cut, curvatures and dimensions of the prepared proximal face 272 and the prepared face 274 match the curvatures and dimensions of the one or more surfaces of the patient glenoid. In an aspect, prior to performance of each of the final width cut and the final depth cut, the method includes determining average curvatures and dimensions of one or more glenoid surfaces for each of a plurality of glenoids from a database, and further includes performing the final width cut and the final depth cut such that the curvatures and dimensions of the prepared proximal face 272 and the prepared face 274 match the average curvatures and dimensions of the one or more glenoid surfaces. As described above with reference to FIGS. 15 and 16, after fitting the prepared graft 250 to the patient glenoid, the fit may be analyzed by measuring the graft 250, performing a trial fit assessment in an open surgical procedure, performing follow-up assessments with the patient after specified periods of time, for example, daily or weekly, or by any suitable assessment methods known in the art or combinations thereof. In an aspect, the method may further include assessing the fit of the distal radius bone graft 250 relative to the glenoid and repeating the steps of positioning the graft 250 in the clamp 200, and performing the final depth cut and/or performing the final width cut as necessary to revise the fit.

In still another alternative, graft 250 could remain in securing tool 210, and securing tool 210 could be detached from base 230, though in this instance fulcrum connector 222 (i.e., the element connecting tool 210 to base 230) should of course be separate from an element, such as a pin, that connects securement features and ends 212, 214, 216, 218 together such that tool 210 remains intact upon separation from base 230.

Although the invention herein has been described with reference to particular aspects, it is to be understood that these aspects are merely illustrative of the principles and applications of the present invention. It is therefore to be understood that numerous modifications may be made to the illustrative aspects and that other arrangements may be devised without departing from the spirit and scope of the present invention as defined by the appended claims.

What is claimed is:

1. A method of preparing a distal radius bone graft comprising:
obtaining the distal radius bone graft;
positioning the distal radius bone graft into a clamp such that a first surface of the distal radius bone graft is removably coupled to a graft-engaging surface of the clamp and at least part of a proximal portion and at least part of a portion opposite the first surface of the distal radius bone graft are accessible;
performing a depth cut on and approximately parallel to the proximal portion of the graft to form a prepared proximal face of the graft; and
performing a width cut on and approximately parallel to the portion opposite the first surface of the graft to form a prepared face of the graft.

2. The method of claim 1, further comprising, prior to at least performing the depth cut and/or performing the width cut, performing a rough cut on at least part of the graft to separate the graft from a remainder of a distal radius and to form a rough face of the graft.

3. The method of claim 1, wherein the method further comprises, after positioning the distal radius bone graft into the clamp, and prior to performing the width cut:
removing the graft from the clamp; and
repositioning the graft into the clamp such that the first surface of the graft is removably coupled to the graft-engaging surface of the clamp such that the graft is flipped to expose a distal face of the graft to a surgical operator.

4. The method of claim 1, wherein the method further comprises, prior to performing the depth cut and performing the width cut, determining curvatures and dimensions of one or more surfaces of a patient glenoid such that following performing the depth cut and performing the width cut, curvatures and dimensions of the prepared proximal face and the prepared face match the curvatures and dimensions of the one or more surfaces of the patient glenoid.

5. The method of claim 1, wherein prior to positioning the distal radius bone graft into the clamp, performing the depth cut and performing the width cut, the method further comprises determining average curvatures and dimensions of one or more graft surfaces from a database,
wherein positioning the distal radius bone graft into the clamp, performing the depth cut and performing the width cut are each performed with the clamp comprising a graft-engaging surface including mating features based on the average curvatures and dimensions of the one or more graft surfaces.

6. The method of claim 5, wherein the graft-engaging surface comprises a plurality of modular inserts configured to match the average curvatures and dimensions of the one or more graft surfaces.

7. The method of claim 1, wherein the clamp includes a drilling guide, the method further comprising drilling a plurality of channels into the graft through a plurality of channels of the drilling guide, each channel within the graft being aligned to a channel of the drilling guide.

8. The method of claim 7, wherein the clamp further comprises a graft-engaging block comprising:
a graft-engaging surface configured to engage the first surface of the graft and secure the graft within the clamp, and
a plurality of channels, each channel of the graft-engaging block being aligned to a channel of the drilling guide,
wherein positioning the distal radius bone graft into a clamp further comprises engaging the first surface of the graft with the graft-engaging surface to secure the graft within the clamp.

9. The method of claim 7, further comprising, prior to the drilling, positioning the drilling guide against the portion opposite the first surface of the graft.

10. The method of claim 1, wherein each of performing the depth cut and performing the width cut is performed while the graft is positioned in the clamp.

11. A clamp system for the preparation of a distal radius bone graft, comprising:
a clamp configured to contact the graft and secure the graft within the clamp; and
a base connected to the clamp, the base comprising a graft-engaging block;
wherein the graft-engaging block comprises a graft-engaging surface configured to engage a first surface of the graft and secure the graft within the clamp,
wherein the clamp is configured to continuously contact and secure the graft while at least a portion of the graft opposite the first surface is removed from the graft.

12. The clamp system of claim 11, wherein the clamp comprises a first securement feature and a second securement feature configured to contact and secure the graft, wherein in a first configuration, the first securement feature is configured to contact a scaphoid facet of the graft and the second securement feature is configured to contact a lunate facet of the graft, and wherein in a second configuration, the first securement feature is configured to contact the lunate facet of the graft and the second securement feature is configured to contact the scaphoid facet of the graft.

13. The clamp system of claim 12, wherein the clamp further comprises:
a first end connected to the first securement feature;
a second end connected to the second securement feature; and
a fulcrum housing connecting the first end and first securement feature to the second end and second securement feature,
wherein the first end and the second end are configured such that when the first end and the second end are moved toward one another, the first securement feature and the second securement feature are moved toward one another, and vice versa.

14. The clamp system of claim 11, wherein the graft-engaging surface comprises a V-block shape, a shape configured to match a three-dimensional shape of a first surface of the graft, or a shape including mating features based on average curvatures and dimensions of one or more graft surfaces determined from a database.

15. The clamp system of claim 11, wherein the graft-engaging surface comprises a plurality of modular inserts configured to match the average curvatures and dimensions of the one or more graft surfaces.

16. The clamp system of claim 11, wherein the base further comprises a drilling guide comprising a plurality of guide channels, the drilling guide being configured to drill a plurality of channels through the graft when a drill bit is passed through the plurality of guide channels and forcibly pressed against the graft.

17. The clamp system of claim 11, wherein the clamp comprises a first securement feature and a second securement feature configured to continuously contact and secure the graft, without contacting a portion opposite the first surface of the graft such that at least part of the portion opposite the first surface of the graft may be removed without physical interference from the first securement feature and the second securement feature.

18. A method of preparing a distal radius bone graft comprising:
obtaining the distal radius bone graft;
positioning the distal radius bone graft into a clamp such that a first surface of the distal radius bone graft is removably coupled to a graft-engaging surface of the clamp and at least part of a proximal portion and at least part of a portion opposite the first surface of the distal radius bone graft is accessible;
performing a depth cut on and approximately parallel to the proximal portion of the graft to form a prepared proximal face of the graft;
removing the graft from the clamp;
repositioning the graft into the clamp such that the first surface of the graft is removably coupled to the graft-engaging surface of the clamp such that the graft is flipped to expose a distal face of the graft to a surgical operator; and
performing a width cut on and approximately parallel to the portion opposite the first surface of the graft to form a prepared face of the graft.

19. The method of claim 18, wherein the clamp comprises a first securement feature and a second securement feature,
wherein removing the graft from the clamp comprises disengaging each of the first securement feature and the second securement feature from the graft, and
wherein repositioning the graft into the clamp comprises engaging each of the first securement feature and the second securement feature with the graft to secure the graft within the clamp.

20. The method of claim 19, wherein:
prior to removing the graft from the clamp, the first securement feature contacts the graft at a scaphoid facet and the second securement feature contacts the graft at a lunate facet, and
after repositioning the graft into the clamp, the first securement feature contacts the graft at the lunate facet and the second securement feature contacts the graft at the scaphoid facet.
